(19) United States
(12) Patent Application Publication
(10) Pub. No.: US 2025/0268825 A1
(43) Pub. Date: Aug. 28, 2025

(54) DELIVERY SYSTEM USING ENGINEERED EXTRACELLULAR VESICLES

(71) Applicant: THE JOHNS HOPKINS UNIVERSITY, Baltimore, MD (US)

(72) Inventors: Guanshu Liu, Baltimore, MD (US); Xiaoning Han, Baltimore, MD (US); Safiya Aafreen, Baltimore, MD (US); Wenshen Wang, Baltimore, MD (US)

(21) Appl. No.: 18/927,448

(22) Filed: Oct. 25, 2024

Related U.S. Application Data
(60) Provisional application No. 63/557,354, filed on Feb. 23, 2024.

(51) Int. Cl. A61K 9/127; A61K 38/20; A61K 45/06; A61K 49/00; A61P 29/00

(52) U.S. Cl. CPC A61K 9/127; A61K 38/2066; A61K 45/06; A61K 49/0084; A61P 29/00

(57) ABSTRACT

The present invention relates to a chimeric extracellular vesicle including a stem cell derived extracellular vesicle (SC-EV) and a liposome containing a therapeutic agent. The SC-EV, derived from stem cells, provides a carrier for the delivery of therapeutic agents. The liposome encapsulates a therapeutic agent, ensuring its stability and controlled release. The combination of SC-EV and liposome allows for targeted and efficient delivery of the therapeutic and imaging agents to specific cells or tissues, enhancing its therapeutic efficacy.

### CROSS-REFERENCE TO RELATED APPLICATIONS

This application claims benefit of priority under 35 U.S.C. § 119(e) of U.S. Provisional Application No. 63/557,354, filed Feb. 23, 2024. The disclosure of the prior application is considered part of and are herein incorporated by reference in the disclosure of this application in their entirety.

### BACKGROUND OF THE INVENTION

### Field of the Invention

The present invention relates generally to delivery vehicles and more specifically to chimeric extracellular vesicles.

### Background Information

Intracerebral hemorrhage (ICH) is the second most common cause of stroke following ischemic stroke, accounting for 27.9% of all new strokes in 2019 globally. Among all strokes, ICH has the highest mortality rate, with early-term mortality of about 30% to 40%, and the highest disability rate, with approximately 70% of patients experiencing long-term deficits. ICH causes brain injury first by hematoma formation, and expansion ensues from initial bleeding, causing mechanical damage (mass effect) to surrounding brain tissue. After the primary injury, secondary injury follows due to perihemorrhagic inflammation, toxic products of blood breakdown, and perihematomal edema, continuing to cause brain injury and neuronal death. The endogenous hematoma clearance system is activated after ICH, mainly through the phagocytosis of microglia/macrophages (M/MΦs). Within one hour after ICH, microglia are activated in response to hematoma. Monocytes in circulation are recruited and mature into macrophages in the brain several hours after ICH. Hence, developing drugs and biologics that can modulate M/MΦs, especially convert them from pro-inflammatory to regulatory phenotypes, is central to discovery of new effective therapy for ICH. Despite tremendous effort devoted to developing treatments aiming to reduce hematoma volume and thereby limit both primary and secondary brain injury after ICH, there is no effective clinically available treatment yet. Surgical treatments, including hematoma evacuation and thrombolytic irrigation have failed in the phase of clinical trials. New medicinal treatments that effectively limit hematoma expansion and accelerate hematoma resolution are desperately needed.

Human stem cells (hSC)-based regenerative medicine has become an increasingly attractive therapeutic approach for ICH. hSCs, including human mesenchymal stem cells (hMSCs), induced pluripotent stem cells (iPSCs) and neural stem cells (NSCs), can effectively protect the impaired brain and improve and recover neurological functions through various mechanisms, including anti-inflammation, anti-apoptosis, neurogenesis/synaptogenesis, angiogenesis, and synaptic plasticity. While hMSCs are being extensively tested in human clinical trials, cell-based therapy has several drawbacks, including biosafety (infusional toxicity, ectopic tissue formation, and tumorigenesis), immunogenicity, and potential ethical concerns, which have led to an increasingly strong interest to develop extracellular vesicles (EVs)-based, cell-free therapies. EVs, often also referred as exosomes in the literature, are small membranous blebs or vesicles released from almost all cell types, serving as mediators of intercellular communication to regulate a diverse range of biological processes. As components of the stem cell secretome, human stem cell derived-EVs (hSC-EVs) have been shown to have great regenerative potential (similar to their parental cells, if not identical) in various diseases including ischemic and hemorrhagic stroke. As a cell-free substitute of stem cell therapy, hSC-EVs provides highly desirable safety and ethical advantages than their parental cells. However, most SC-EV-related studies done till now are proof-of-concept without rigorous quality control and validation and often lack technical optimization. Recent studies have shown that hMSC-EVs are particularly effective as immunomodulatory therapy.

A classic anti-inflammatory cytokine, interleukin-10 (IL-10), can exert substantial neuroprotection in ICH by eliciting M/MΦ phagocytosis function and anti-inflammatory polarization. In the central nervous system (CNS), IL-10 is primarily produced by microglia. Microglia-derived IL-10 accelerated post-ICH hematoma clearance through the signal transducer and activator of transcription 3 (STAT3)-dependent pathway. However, significantly improved outcomes have not been obtained if IL-10 is directly administered systemically, indicating suitable drug delivery methods are required for using IL-10 to treat human diseases. It is particularly a problem for ICH, in which blood-brain barrier (BBB) exists as formidable technical hurdle preventing an adequate amount of IL-10 to reach the perihematomal regions.

Intravenously (i.v.) administered IL-10 suffers from short half-life and may lead to unwanted effects on other immune cells. Recently, intranasal (i.n.) injection emerges as a minimally invasive route for drug delivery to the brain. Rather than crossing the BBB from the blood circulation (like intravenous injection), intranasally injected nanoparticles cross the nasal epithelium via the transcellular pathway (e.g., axonal transport) or paracellular pathway (or the “nose-brain barrier” and are transported along the olfactory and trigeminal nerves). Thus, intranasal delivery is the simpler and direct way for brain targeting, which avoids bloodstream clearance and invasive methods. However, obtaining consistent and satisfactory delivery efficacies of drugs administered intranasally, whether in their free form or encapsulated within synthetic nanoparticles, remains a challenging task.

The present invention addresses multiple challenges faced in the treatment of inflammatory diseases, especially neuroinflammatory conditions. Conventional treatments often suffer from non-specific targeting, leading to off-target effects and reduced therapeutic efficiency. This present invention optimizes drug delivery by ensuring that therapeutics reach the intended inflammatory sites with precision. Many contemporary solutions raise concerns over biocompatibility, potentially causing adverse reactions. By harnessing human stem cell-derived extracellular vesicles, the present disclosure describes a design that prioritizes biocompatibility, reducing the risk of unfavorable host responses. The unmet need for a targeted, effective, biocompatible treatment solution with real-time monitoring capability is vast. In 2020, the global market for theranostics was valued at $80.4 billion, and that for intracerebral hemorrhage was $670 million.

Fortunately, mounting evidence has shown that EVs have an ability to transport biomolecules among cells across various biological barriers, including BBB, offering an advantage in CNS applications. Many EV-based nanoparticle drug delivery systems are shown to be more effective than their synthetic counterparts. Moreover, once reaching the recipient cells, EVs are taken up by cells via various endocytosis pathways, in a more efficient way than synthetic nanoparticles, making them more suited for delivering therapeutic agents that act on intracellular compartments or organelles. Those properties collectively make EVs a superior drug delivery platform.

### SUMMARY OF THE INVENTION

The present disclosure is based on the surprising discovery that fusion of EVs and liposomes, coupled with a cytokine, e.g., IL-10, leads to improved therapeutic and diagnostic efficacy. This synergy amplifies the medicinal effects of cytokine or immunomodulatory agent therapy, especially when compared to systems that deploy these components in isolation. Current treatment modalities rarely offer simultaneous therapeutic intervention and diagnostic imaging. The present disclosure describes therapeutic agents that are both therapeutic and include imaging agents, monitoring of therapeutic effects in real-time, and rapid adjustment of therapeutic treatments.

In one embodiment, the present disclosure relates to a stem cell derived extracellular vesicle (SC-EV); and a liposome including a therapeutic agent. In one aspect, the liposome includes at least one of phosphatidylcholine or phosphatidylserine. In one aspect, the liposome is a phosphatidylserine (PS) liposome. In one aspect, the stem cell is a mesenchymal stem cell (MSC), a hematopoietic stem cell (HSC), an induced pluripotent stem cell (iPSC), an adipose tissue derived stem cell, or a neural stem cell (NSC). In one aspect, the therapeutic agent is a cytokine, an immunomodulatory agent, an antioxidant, an miRNA, or an antibody. In one aspect, the cytokine is interleukin-1 (IL-1), interleukin-2 (IL-2), interleukin-6 (IL-6), interleukin-10 (IL-10), interleukin-12 (IL-12), interleukin-15 (IL-15), interleukin-18 (IL-18), tumor necrosis factors (TNF), interferons (IFN), colony-stimulating factors (GM-CSF) or transforming growth factor-β (TGF-β). In one aspect, the immunomodulatory agent is a checkpoint inhibitor, a small molecule, or a chimeric antigen receptor. In one aspect, the antibody is adalimumab, infliximab, certolizumab, golimumab, or tocilizumab. In one aspect, the liposome further includes an imaging agent. In one aspect, the imaging agent is selected from superparamagnetic iron oxide (SPIO) nanoparticles, a lipid conjugated metal chelator, a radionucleotide, a fluorescent protein or a lipophilic fluorescence dye. In one aspect, the composition further includes a pharmaceutically acceptable carrier.

In one embodiment, the present disclosure relates to a method of generating a chimeric extracellular vesicle (cEV) including fusing a stem cell derived extracellular vesicle (SC-EV) and a liposome including a therapeutic agent, thereby generating a cEV. In one aspect, the liposome includes at least one of phosphatidylcholine or phosphatidylserine. In one aspect, the liposome is a phosphatidylserine (PS) liposome. In one aspect, the stem cell is a mesenchymal stem cell (MSC), a hematopoietic stem cell (HSC), an induced pluripotent stem cell (iPSC), an adipose tissue derived stem cell, or a neural stem cell (NSC). In one aspect, the therapeutic agent is a cytokine, an immunomodulatory agent, an antioxidant, an miRNA, or an antibody. In one aspect, the cytokine is interleukin-1 (IL-1), interleukin-2 (IL-2), interleukin-6 (IL-6), interleukin-10 (IL-10), interleukin-12 (IL-12), interleukin-15 (IL-15), interleukin-18 (IL-18), tumor necrosis factors (TNF), interferons (IFN), colony-stimulating factors (GM-CSF) or transforming growth factor-β (TGF-β). In one aspect, the immunomodulatory agent is a checkpoint inhibitor, a small molecule, or a chimeric antigen receptor. In one aspect, the antibody is adalimumab, infliximab, certolizumab, golimumab, or tocilizumab. In one aspect, the liposome further includes an imaging agent. In one aspect, the imaging agent is selected from superparamagnetic iron oxide (SPIO) nanoparticles, a lipid conjugated metal chelator, a radionucleotide, a fluorescent protein or a lipophilic fluorescence dye.

In one embodiment, the techniques described herein relate to a method of treating inflammation including administering the composition described herein to a subject in need thereof. In one aspect, the inflammation is neuroinflammation. In one aspect, the subject has an intracerebral hemorrhage (ICH). In one aspect, the administering is systemic, intranasal, intrathecal or intracerebral.

In one embodiment, the present disclosure relates to a method of detecting or guiding cEV-based therapy including administering a composition described herein to a subject and detecting the composition after administration. In one aspect, the detecting is by magnetic particle imaging (MPI), positron emission tomography (PET), fluorescence imaging (FI) or bioluminescence imaging (BLI). In one aspect, the detecting provides information for optimization of the cEV-based therapy.

### BRIEF DESCRIPTION OF THE DRAWINGS

FIG. 1 is an illustrative schematic of the design of the new type of ICH therapy by synergizing the advantages of human stem cell-derived EVs and IL-10-carrying PS liposomes.

FIG. 2 is an illustrative schematic of the hybrid EVs that integrate MSC-EVs with IL-10 liposomes, together with a multimodal imaging capability allowing tracking, guiding, and optimizing EV-based therapy.

FIGS. 3A-3D is an illustrative schematic of hybrid extracellular vesicles for treating ICH in a new theranostic manner, in which multimodal imaging is integrated for tracking, guiding, and optimizing. FIG. 3A MRI detection of the biodistribution i.v. injected SPIO-labeled eEV-IL10 (yellow arrows, 5×10⁹ per mouse) in the ipsilateral side of intracranial LPS injection (24 h post) to induce neuroinflammation. FIG. 3B PET images showing the biodistribution of i.v. injected eEV-IL10 (5×10⁹ per mouse) in ICH and healthy control mice. A higher intracranial uptake of eEV-IL10 in the ICH mouse than in the sham control is clearly seen in the zoomed view (right panel) of the mouse brains. FIG. 3C Fluorescence images showing the ability to in vivo dynamic detection of the biodistribution (dorsal view) of intranasal injected eEV-IL10 (6.3×10⁸ per mouse). The hybrid EVs were labeled with IRDye800-labeled FIG. 3D Bioluminescence images of a healthy mouse in prone, supine, and side positions at 10 mins after i.v. administration of 1×10⁹ NLuc-EVs.

FIGS. 4A-4B is a set of images and graphs illustrating the efficacy of eEV-IL10 in reducing injury volume in ICH mice. FIG. 4A. Luxol fast blue/cresyl violet (FB/CV) staining. FIG. 4B. Fluro-Jade C (FJC) staining for neuronal degeneration in the perihematomal regions. Mice were treated with different particles (i.n) at 30 min following ICH. One-way ANOVA analysis followed by Tukey. Sample size: vehicle (n=5), PSL (n=4), PSL-IL-10 (n=4), EV-IL10 (n=3), and PSL-EV-IL10 (n=5). Mean±SD.

FIGS. 5A-5C is a set of images illustrating multimodal imaging for in vivo detection and quantification of hybrid EVs noninvasively. FIG. 5A MRI detection of the biodistribution i.v. injected SPIO-labeled hybrid EVs (yellow arrows, dose=5×10⁹ per mouse) in the perihematomal regions (24 h post-ICH), where the hematoma appeared as hypointensity in the lightly weighted T2w image (left), which is marked by green dashed line in the T2* image to show the co-localization of EVs and hematoma. FIG. 5B PET images showing the biodistribution of i.v. injected hybrid EVs (dose=5×10⁹ per mouse) in ICH and healthy control mice. A higher intracranial uptake of hybrid EVs in the ICH mouse than the sham control are clearly seen in the zoomed view (right panel) of the mouse brains. FIG. 5C Fluorescence images showing the ability to in vivo monitor the delivery of hybrid EVs (left, mouse in dorsal view) after the intranasal injected hybrid EVs (dose=6.3×10⁸ per mouse) and assess the biodistribution by ex vivo FI (left) at 2 hours after i.v. of particles (4×10⁹ per mouse). The results clearly showed the BBB crossing ability of iPSC-EVs and hybrid EVs, but not liposomes. All particles were labeled with IRDye800-CW.

FIGS. 6A-6F is a set of images and graphs illustrating that PSL-IL10 is selectively taken up by M/MΦs after ICH. PSL-IL10 was labeled with Rhodamine B (red) and intranasal administered to ICH mice.

FIG. 6A is a set of images of immunostaining showing M/MΦs (Iba1, green) co-localized with PSL-IL10 (red).

FIG. 6B is a Western blot of IL-10 level post-ICH.

FIG. 6C is a graph showing IL-10 levels at day 3 post-ICH. Two-way ANOVA followed by Tukey. n=4. Mean±SD. *p<0.05 and **p<0.01.

FIG. 6D is a graph showing hematoma size at day 3 post-ICH. Two-way ANOVA followed by Tukey. n=4. Mean±SD. *p<0.05 and **p<0.01.

FIG. 6E is a set of images showing hematoma size at day 1, 3 and 7 post-ICH. Two-way ANOVA followed by Tukey. n=4. Mean±SD. *p<0.05 and **p<0.01.

FIG. 6F is a graph showing hematoma size at day 1, 3 and 7 post-ICH. Two-way ANOVA followed by Tukey. n=4. Mean±SD. *p<0.05 and **p<0.01.

FIG. 7 is an image comparing the distribution of (iPSC-) cEVs by IN and IV injection using in vivo fluorescence imaging at 24 hrs post-administration. EVs were labeled using IRdye800CW as described in the method section.

FIG. 8 is an illustrative schematic of the experimental design.

FIG. 9 is an illustrative schematic showing that efficient drug delivery is required to fully exert the therapeutic effects of IL-10. This approach has been demonstrated using liposomes. The present study is rationalized based on the even higher delivery efficiency of stem cell-derived extracellular vehicles (EVs) to the CNS, which is anticipated to significantly improve the treatment efficacy of IL-10 against ICH.

FIG. 10 is an image and graph of confirmed microglia/macrophage (M/MØ) -targeting of eEV-IL10 in mouse brains after ICH. eEV-IL10 was intracranially injected within 2 hours after collagenase-induced ICH. Representative images show the distribution of eEV-IL10 in the ipsilateral striatum on 3 days after ICH. Upper images: the dash line shows the center of ICH. Green: microglia stained by Iba1; Red: fluorescently labeled eEV-IL10 (by RhoB). Scale bar: 50 μm. Lower images show eEV-IL10 specificity in M/MO under high magnification. Scale bare: 10 μm. Graph shows higher M/MØ-targeting ability of eEV-IL10 in the perihematomal regions after ICH.

FIG. 11 is an illustrative schematic showing use of conditioned cell culture media (CCM). The supernatant of the cell culture media was first centrifuged at 200×g for 5 minutes to remove any floating/dead cells. After the first centrifugation, the supernatant was then centrifuged at 2000×g for 10 minutes to remove any big cell debris. After both rounds of centrifugation, the cell culture media (CCM) was stored at −80° C. until it was thawed overnight at 4° C. before use.

FIG. 12 is a graph of RT-qPCR results revealing the enhanced effect of eEV-IL10 (prepared by iPSC-EVs and PSL-IL10) on the IL-10 mRNA level in LPS/IFN-γ treated RAW264.7 cells.

FIG. 13 is a set of images showing bioparticles (red) phagocytosed by the cultured primary microglia. Both PSL-IL10 (middle) and eEV-IL10 (bottom) promote the microglial phagocytosis. Scale bar=10 μm.

FIG. 14 is a set of graphs of BLI for studying the biodistribution of EVs administered intravenously or intranasally into mice. EV uptake into mouse tissues was determined by NanoGlo assay on tissue homogenates. Datapoints show the amount of nanoluciferase signal (RLU) normalized to the protein content of the lysates. Data from n=5 animals are shown. Statistical comparisons: one-way ANOVA with Tukey's post-hoc test, *p<0.05, **p<0.01, and ***p<0.001.

FIG. 15 is a set of graphs of neurologic deficit behavior test results. All treatments were administered within 2 h after ICH via i.v. injection. Data are shown as box-and-whiskers. One-way or two-way ANOVA post hoc Turkey test were performed. n=4-5 for each group.

### DETAILED DESCRIPTION OF THE INVENTION

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein and in the claims, the terms “comprising,” “containing,” and “including” are inclusive, open-ended and do not exclude additional unrecited elements, compositional components or method steps. Accordingly, the terms “comprising” and “including” encompass the comparably more restrictive terms “consisting of” and “consisting essentially of.”

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

As used herein, the term “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

As used herein the terms “antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. “Antibody,” as used herein, encompasses any polypeptide comprising an antigen-binding site regardless of the source, species of origin, method of production, and characteristics. Antibodies include natural or artificial, mono- or polyvalent antibodies including, but not limited to, polyclonal, monoclonal, multispecific, human, humanized or chimeric antibodies, single chain antibodies, and antibody fragments. “Antibody fragments” include a portion of an intact antibody, preferably the antigen binding or variable region of the intact antibody. Examples of antibody fragments include Fab, Fab′ and F(ab′)2, Fc fragments or Fc-fusion products, single-chain Fvs (scFv), disulfide-linked Fvs (sdfv) and fragments including either a VL or VH domain; diabodies, tribodies and the like (Zapata et al. Protein Eng. 8(10):1057-1062 [1995]). The term “antibody,” as used herein, refers to immunoglobulin molecules and immunologically active portions of immunoglobulin molecules, i.e., molecules that contain an antigen binding site that immunospecifically binds an antigen.

Experimentally, in one aspect, antibodies are cleaved with the proteolytic enzyme papain, which causes each of the heavy chains to break, producing three separate antibody fragments. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding but is used in later processes involved in ridding the body of the antigen.

As used herein, the term “antigen” refers to a molecule or entity to which an antibody binds. In some embodiments, an antigen is or comprises a polypeptide or portion thereof. In some embodiments, an antigen is a portion of an infectious agent that is recognized by antibodies. In some embodiments, an antigen is an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In general, an antigen may be or include any chemical entity such as, for example, a small molecule, a nucleic acid, a polypeptide, a carbohydrate, a lipid, a polymer other than a biologic polymer (e.g., other than a nucleic acid or amino acid polymer) etc. In some embodiments, an antigen is or comprises a polypeptide.

As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid is isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be employed for introduction into, i.e., transfection of, cells, in particular, in the form of RNA which is prepared by in vitro transcription from a DNA template. The RNA can moreover be modified before application by stabilizing sequences, capping, and polyadenylation.

Generally, nucleic acid is extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; U.S. Pub. 2010/0285578; and U.S. Pub. 2002/0190663.

Isolated nucleic acid sequences, or alternatively, “codon-optimized” sequences of nucleic acid sequences of interest, modified to provide the sequences with preferred optimized characteristics, are provided. Such characteristics may include transcription, translation, post-translational modification, stability of the encoded protein, etc. When two or more nucleic acid sequences are included in a single vector or construct, they are in operable linkage, such that the sequences are properly encoded and expressed. In some embodiments, an expression vector includes nucleic acid. In some embodiments, a polynucleotide sequence of the nucleic acid encodes a selectable marker (SM) protein that is operably linked to a protein of interest (POI) (e.g., an antibiotic resistance gene). In some embodiments, a nucleic acid is operably linked to an expression control sequence (e.g., a promoter). As used herein, a nucleotide sequence is “operably linked” with an expression control sequence when the expression control sequence functions in a cell to regulate transcription of the nucleotide sequence. This includes promoting transcription of the nucleotide sequence through an interaction between a polymerase and a promoter.

The terms “peptide”, “polypeptide” and “protein” are used interchangeably herein, and refer to any chain of at least two amino acids linked by a covalent chemical bound. As used herein, a peptide can refer to the complete amino acid sequence coding for an entire protein or to a portion thereof. A “protein coding sequence,” or a sequence that “encodes” a particular polypeptide or peptide, is a nucleic acid sequence that is transcribed, in the case of DNA, and is translated, in the case of mRNA, into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence is usually be located 3′ to the coding sequence.

The term “extracellular vesicle” (EV) refers to lipid bilayer-delimited particles that are naturally released from cells. EVs range in diameter from around 20-30 nanometers to about 10 microns or more. EVs can include proteins, nucleic acids, lipids and metabolites from the cells that produced them. EVs include primarily exosomes, which are about 30 to about 150 nm, have the same topology and are enriched in exosome marker proteins, and also larger EVs such as microvesicles that are greater than 200 nm, but usually less than 500 nm. Preparations of small EVs and exosomes and large EVs and microvesicles are often contaminated by extracellular particles (EPs). EPs lack a membrane bilayer but in one aspect, they are of similar size to EVs. Common EPs include lipoprotein particles, exomeres, non-enveloped virus particles, and aggregated protein particles released by necrotic cells.

In one aspect, extracellular vesicles are referred to herein as “delivery vehicles.” In one aspect, an extracellular vesicle carries a cargo, which, in one aspect, is a protein of interest (POI) or a nucleic acid of interest (NAOI). In one aspect, the cargo molecule is present within the lumen of the delivery vehicle. In another aspect, the cargo molecule is on the surface of the delivery vehicle. In one aspect, the protein of interest is a protein naturally produced by a cell that generates a delivery vehicle. In another aspect, the protein of interest is a recombinant protein, including a non-naturally occurring protein. In one aspect, the protein of interest is a fusion protein. In one aspect, the POI is a viral protein, e.g., capable of eliciting an immune response. Nucleic acids include, without limitation, DNA and RNA. In one aspect, RNA is mRNA. In one aspect, when delivered to a target cell, mRNA is expressed as protein and presented on the cell surface to elicit an immune response. Nucleic acids are typically incorporated into EVs by contacting the EVs and the nucleic acid in the presence of a chemical lipofection reagent. In one aspect, the chemical lipofection reagent is a polycationic lipid. In some embodiments, the chemical lipofection reagent is an mRNA lipofection reagent or an mRNA transfection reagent; for example, Lipofectamine® MessengerMAX™, Lipofectamine® 2000, Lipofectamine® 3000.

Exosomes are defined herein as all small, secreted vesicles of approximately 20-150 nm that are released by mammalian cells and made either by budding into endosomes or by budding from the plasma membrane of a cell. Exosomes can range in size from approximately 20-150 nm in diameter. In some cases, they have a characteristic buoyant density of approximately 1.1-1.2 g/mL, and a characteristic lipid composition. Their lipid membrane is typically rich in cholesterol and contains sphingomyelin, ceramide, lipid rafts and exposed phosphatidylserine. Exosomes express certain marker proteins, such as integrins and cell adhesion molecules, but generally lack markers of lysosomes, mitochondria, or caveolae. In some embodiments, the exosomes contain cell-derived components, such as but not limited to, proteins, DNA, and RNA (e.g., microRNA and noncoding RNA). In some embodiments, exosomes are obtained from cells obtained from a source that is allogeneic, autologous, xenogeneic, or syngeneic with respect to the recipient of the exosomes.

Exosomes are collected, concentrated and/or purified using methods known in the art. For example, differential centrifugation has become a leading technique wherein secreted exosomes are isolated from the supernatants of cultured cells. This approach allows for separation of exosomes from larger extracellular vesicles and from most non-particulate contaminants by exploiting their size. Exosomes are prepared as described in a wide array of papers, including but not limited to, Fordjour et al., “A shared pathway of exosome biogenesis operates at plasma and endosome membranes”, bioRxiv, preprint posted Feb. 11, 2019, at https://www.biorxiv.org/content/10.1101/545228v1; Booth et al., “Exosomes and HIV Gag bud from endosome-like domains of the T cell plasma membrane”, J Cell Biol., 172:923-935 (2006); and, Fang et al., “Higher-order oligomerization targets plasma membrane proteins and HIV gag to exosomes”, PLoS Biol., 5:e158 (2007). Exosomes using a commercial kit such as, but not limited to the ExoSpin™ Exosome Purification Kit, Invitrogen® Total Exosome Purification Kit, PureExo® Exosome Isolation Kit, and ExoCap™ Exosome Isolation kit. Methods for isolating exosome from stem cells are found in, e.g., Tan et al., Journal of Extracellular Vesicles, 2:22614 (2013); Ono et al., Sci Signal, 7(332):ra63 (2014) and U.S. Application Publication Nos. 2012/0093885 and 2014/0004601. Methods for isolating exosome from cardiosphere-derived cells are found in, e.g., Ibrahim et al., Exosomes as important agents of cardiac regeneration triggered by cell therapy, Stem Cell Reports, 2014. Specific methodologies include ultracentrifugation, density gradient, HPLC, adherence to substrate based on affinity, or filtration based on size exclusion. The term “vector”, “expression vector”, or “plasmid DNA” is used herein to refer to a recombinant nucleic acid construct that is manipulated by human intervention. A recombinant nucleic acid construct can contain two or more nucleotide sequences that are linked in a manner such that the product is not found in a cell in nature. In particular, the two or more nucleotide sequences are operatively linked, such as one or more genes encoding one or more proteins of interest, one or more protein tags, functional domains and the like. The expression vector of the invention can include regulatory elements controlling transcription generally derived from mammalian, microbial, viral or insect genes, such as an origin of replication, to confer to the vector the ability to replicate in a host, and a selection gene may additionally be incorporated, encoding, for example, a selectable marker (SM) protein to facilitate recognition of transformants. Those of skill in the art can select a suitable regulatory region to be included in such a vector, depending on the host cell used to express the gene(s). For example, the expression vector can include one or more promoters, operably linked to the nucleic acid of interest, or a gene of interest (GOI) capable of facilitating transcription of genes in operable linkage with the promoter. Several types of promoters are well known in the art and suitable for use with the present invention. In one aspect, the promoter is constitutive. In one aspect, the promoter is inducible.

Additional regulatory elements that may be useful in vectors include, but are not limited to, polyadenylation sequences, translation control sequences (e.g., an internal ribosome entry segment, IRES), enhancers, introns, and the like. Such elements may not be necessary, although they may increase expression by affecting transcription, stability of the mRNA, translational efficiency, and the like. Such elements are included in a nucleic acid construct as desired to obtain high expression of the nucleic acids in the cell(s). Sufficient expression, however, may sometimes be obtained without such additional elements. Vectors also can include other elements. For example, a vector can include a nucleic acid that encodes a signal peptide such that the encoded polypeptide is directed to a particular cellular location (e.g., a signal secretion sequence to cause the protein to be secreted by the cell), or it can include a nucleic acid that encodes a selectable marker. Non-limiting examples of selectable markers include doxycycline, puromycin, adenosine deaminase (ADA), aminoglycoside phosphotransferase (neo, G418, APH), dihydrofolate reductase (DHFR), hygromycin-B-phosphotransferase, thymidine kinase (TK), and xanthin-guanine phosphoribosyltransferase (XGPRT). Such markers are useful for selecting stable transformants in culture. Non-limiting examples of vectors suitable for use for the expression of high levels of recombinant proteins of interest include those selected from baculovirus, phage, plasmid, phagemid, cosmid, fosmid, bacterial artificial chromosome, viral DNA, P1-based artificial chromosome, yeast plasmid, transposon, and yeast artificial chromosome. For example, in one aspect, the viral DNA vector is selected from vaccinia, adenovirus, foul pox virus, pseudorabies and a derivative of SV40. Non-limiting examples of suitable bacterial vectors include pQE70™, pQE60™, pQE-9™, pBLUESCRIPT™ SK, pBLUESCRIPT™ KS, pTRC99a™, pKK223-3™, pDR540™, PAC™ and pRIT2T™. Non-limiting examples of suitable eukaryotic vectors include pWLNEO™, pXTI™, pSG5™, pSVK3™, pBPV™, pMSG™, and pSVLSV40™. Non-limiting examples of suitable eukaryotic vectors include pWLNEO™, pXTI™, pSG5™, pSVK3™, pBPV™, pMSG™, and pSVLSV40™. In one aspect, one type of vector is a genomic integrated vector which is integrated into the chromosomal DNA of the host cell. Another type of vector is an episomal vector, e.g., a nucleic acid capable of extra-chromosomal replication. Viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). In one aspect, viral vectors are modified so the native tropism and pathogenicity of the virus are altered or removed. In another aspect, the genome of a virus is modified to increase its infectivity and to accommodate packaging of the nucleic acid encoding the polypeptide of interest.

Other non-limiting examples of vectors, suitable for use for the expression of high levels of recombinant proteins of interest include those selected from baculovirus, phage, plasmid, phagemid, cosmid, fosmid, bacterial artificial chromosome, viral DNA, P1-based artificial chromosome, yeast plasmid, transposon, and yeast artificial chromosome. For example, the viral DNA vector is selected from vaccinia, adenovirus, foul pox virus, pseudorabies and a derivative of SV40. Suitable bacterial vectors for use in practice of the invention methods include pCG473, pCG512, pCG546, pCG550, pCG552, pJM1463, pJM1464, pQE70™, pQE60™, pQE-9™ pBLUESCRIPT™ SK, pBLUESCRIPT™ KS, pTRC99a™, pKK223-3™, pDR540, PAC™ and pRIT2T™. Suitable eukaryotic vectors for use in practice of the invention methods include pWLNEO™, pXTI™, pSG5™, pSVK3™, pBPV™, pMSG™, and pSVLSV40™. Suitable eukaryotic vectors for use in practice of the invention methods include pWLNEO™, pXTI™, pSG5™, pSVK3™, pBPV™, pMSG™, and pSVLSV40™. One type of vector is a genomic integrated vector, or “integrated vector,” which is integrated into the chromosomal DNA of the host cell. Another type of vector is an episomal vector, e.g., a nucleic acid capable of extra-chromosomal replication. Viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors are modified so that the native tropism and pathogenicity of the virus has been altered or removed. The genome of a virus also is modified to increase its infectivity and to accommodate packaging of the nucleic acid encoding the polypeptide of interest.

The nucleic acid construct (or the vector) of the present invention may be introduced into a host cell to be altered thus allowing expression of the protein within the cell. A variety of host cells are known in the art and suitable for proteins expression and extracellular vesicles production. Examples of typical cell used for transfection include, but are not limited to, a bacterial cell, a eukaryotic cell, a yeast cell, an insect cell, or a plant cell. For example, Human embryonic kidney 293 (HEK293), E. coli, Bacillus, Streptomyces, Pichia pastoris, Salmonella typhimurium, Drosophila S2, Spodoptera SJ9, CHO, COS (e.g., COS-7), 3T3-F442A, HeLa, HUVEC, HUAEC, NIH 3T3, Jurkat, FtetZ/CG473, FtetZ/293F, 293, 293F, F/YA24, 293H, HEK293, or 293F.

A variety of methods are known in the art and are suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as LIPOFECTAMINE™, DOJINDO HILYMAX™, FUGENE™, JETPEI™, EFFECTENE™ and DREAMFECT™.

The terms “treat”, “therapeutic”, “prophylactic” and “prevent” are not intended to be absolute terms. Treatment, prevention and prophylaxis can refer to any delay in onset, amelioration of symptoms, improvement in patient survival, increase in survival time or rate, etc. Treatment, prevention, and prophylaxis is complete or partial. The term “prophylactic” means not only “prevent”, but also reduce illness and disease. For example, a “prophylactic” agent is administered to a subject, e.g., a human subject, to prevent infection, or to lower the extent of illness and disease caused by such infection. The effect of treatment is compared to an individual or pool of individuals not receiving the treatment, or to the same patient prior to treatment or at a different time during treatment. In one aspect, the severity of disease is reduced by at least 10%, as compared, e.g., to the individual before administration or to a control individual not undergoing treatment. In one aspect, the severity of disease is reduced by at least 25%, 50%, 75%, 80%, or 90%, or in some cases, no longer detectable using standard diagnostic techniques.

A treatment is considered “effective,” as used herein, if one or more of the signs or symptoms of a condition described herein are altered in a beneficial manner, other clinically accepted symptoms are improved, or even ameliorated, or a desired response is induced e.g., by at least 2%, 3%, 4%, 5%, 10%, or more, following treatment according to the methods described herein. Efficacy is assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (e.g., progression of the disease is halted). Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent is determined by assessing physical indicators of a condition or desired response. One skilled in the art can monitor efficacy of administration and/or treatment by measuring any one of such parameters, or any combination of parameters.

The term “effective amount” as used herein refers to the amount of a composition or an agent needed to alleviate at least one or more symptom of the disease or disorder and relates to a sufficient amount of therapeutic composition to provide the desired effect. The term “therapeutically effective amount” refers to an amount of a composition or therapeutic agent that is sufficient to provide a particular effect when administered to a typical subject. An effective amount as used herein, in various contexts, can include an amount sufficient to delay the development of a symptom of the disease, alter the course of a symptom disease (for example but not limited to, slowing the progression of a symptom of the disease), or reverse a symptom of the disease. For example, for the given parameter, a therapeutically effective amount shows an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control. The therapeutically effective amount may be administered in one or more doses of the therapeutic agent. The therapeutically effective amount may be administered in a single administration, or over a period of time in a plurality of doses.

“Administering” as used herein can include any suitable routes of administering a therapeutic agent or composition as disclosed herein. Suitable routes of administration include, without limitation, intranasal, oral, parenteral, intravenous, intraventricular, intramuscular, subcutaneous, transdermal, airway (aerosol), pulmonary, cutaneous, injection or topical administration. Administration is local or systemic.

As used herein, the term “pharmaceutically acceptable” refers to a carrier that is compatible with the other ingredients of the formulation and not deleterious to the recipient thereof. The term is used synonymously with “physiologically acceptable” and “pharmacologically acceptable”. A pharmaceutical composition comprises agents for buffering and preservation in storage, and in one aspect, the composition includes buffers and carriers for appropriate delivery, depending on the route of administration. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

This invention represents a ground-breaking improvement over current technologies. While there are existing methods and products for treating inflammatory diseases, including neuroinflammatory conditions, the IL-10 carrying engineered EVs offer a combination of targeted drug delivery and theranostic capabilities. This dual function, both therapeutic and diagnostic, positions it as a transformative advancement in the field.

Synergy of Components: The engineered EV formulation combines the natural targeting and biocompatibility properties of EVs with the drug-loading capacity of liposomes, maximizing the therapeutic potential of IL-10.

Targeted Delivery: By leveraging the targeting capabilities of EVs, the engineered form ensures that IL-10 is delivered precisely to the sites of inflammation, lowering systemic side effects commonly seen with other treatments.

Theranostic Capabilities: Beyond merely treating the condition, engineered EVs integrate various imaging agents, allowing for real-time monitoring of drug delivery and therapeutic response. This capability is rarely found in many of the current treatment methods, offering clinicians an unprecedented tool to adjust treatment protocols in real-time based on patient response.

Broad Applicability: The platform's versatility means it could be adapted not just for neuroinflammatory conditions, but for a wide range of inflammatory diseases. This broadens its potential patient impact considerably.

This technology encompasses an innovative approach to the design and utilization of extracellular vesicles (EVs) as a multifunctional therapeutic and diagnostic tool. Central to this invention is the formation of an IL-10 carrying engineered extracellular vesicles (EVs), a harmonious amalgamation of three principal constituents:
a) Human Stem Cell-Derived Extracellular Vesicles (EVs): These bio-nanoscale vesicles are known for their biocompatibility and adaptability, promoting intercellular communication and potentially assisting in tissue regeneration across diverse inflammatory scenarios.
b) IL-10 Loading through membrane fusion: Surface-modified with the powerful anti-inflammatory cytokine, Interleukin-10 (IL-10), phosphatidylserine liposomes are utilized to incorporate a sufficient amount of IL-10 to the out layer of EVs, namely engineered EVs, which are released in a targeted and prolonged manner at inflammation sites, enhancing its therapeutic efficacy.
c) Imaging Capacity: in some aspects, this engineered EV formulation is further equipped with molecular imaging agents, facilitating real-time visualization of vesicle distribution, gauging therapeutic efficiency, and tracking disease evolution.

Through their integrated design, engineered EVs offer a dual solution: they not only therapeutically modulate inflammation but also concurrently provide diagnostic imaging insight. The developed engineered EVs have higher inflammation targeting and great applicability to a broad range of inflammatory diseases, including those with a neuroinflammatory component.

The IL-10 carrying engineered EVs are a type of theranostic nanomedicine: A singular product that offers both therapeutic and diagnostic capabilities, useful for clinicians who monitor treatment outcomes in real-time and adapt treatment strategies promptly based on immediate feedback. The hybrid EVs are a modular drug delivery system, given the customizable design. In one aspect, different therapeutic entities or imaging tags are interchanged based on specific patient needs or disease conditions. For chronic inflammatory diseases, including neuroinflammatory conditions, specialized kits are formulated. These kits contain engineered EVs tailored for specific conditions, ensuring that patients receive optimized treatment for their condition. With the capability to host molecular imaging agents, the hybrid EV is also a diagnostic tool, especially for conditions where real-time monitoring of inflammation or tissue regeneration is important.

The overall goal of this project is to develop a new regenerative therapy for effectively improving outcomes following intracerebral hemorrhage (ICH). The immunomodulatory effects and blood-brain barrier (BBB) penetrating ability of human mesenchymal stem cells (hMSC) derived extracellular vesicles (EVs) using drug-carrying, microglia/macrophage (M/MΦ)-targeted nanotherapeutics for optimized attenuation of ICH-induced inflammatory injury and hematoma-caused damage. Compared to single therapy alone, the new, hMSC-EVs-based therapy is unprecedented and results in a significantly improved outcome following ICH, paving a new pathway to reduce mortality and disability rate in ICH patients, which remains an unmet clinical need.

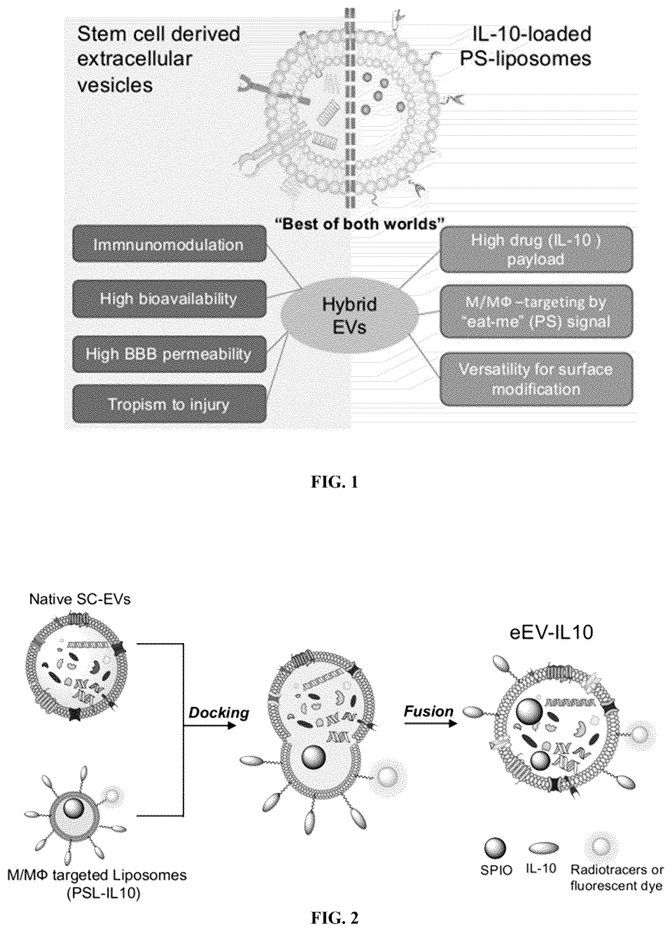

FIG. 1 depicts the presently described approach to develop a novel therapeutic strategy for ICH by combining the advantages of human stem cell-derived extracellular vesicles (EVs) and interleukin-10 (IL-10) carrying phosphatidylserine (PS) liposomes, referred to as chimeric EVs (cEVs) or hybrid EVs. Because the functionality of M/MΦ is one of the factors affecting the outcome of ICH, by utilizing immunomodulatory SC-EVs and M/MΦ-targeted liposomes, cEVs are engineered for effective brain delivery to transport therapeutic drugs to M/MΦ in the perihematomal regions, leading to a substantial improvement in the outcome of ICH. Multimodal imaging capabilities are incorporated in the design so that non-invasive imaging technology is utilized not only for preclinical optimization but also for future clinical applications. A proprietary MRI method was developed for tracking EVs in vivo, which in one aspect is combined with magnetic particle imaging (MPI), positron emission tomography (PET), fluorescence imaging (FI), and bioluminescence imaging (BLI) to enable multimodal tracking, guiding, and optimization of cEV-based ICH therapy. This theranostic system is novel and innovative.

In this study, EV-based delivery strategy is used to solve this unmet clinical need. EVs are more powerful for drug delivery than synthetic counterparts because of the ability of EVs to transport biomolecules between cells. Regardless of the difference in the whole-body distribution of EVs derived from different cell origins, EVs have an advantage for CNS applications by their ability to cross BBB for effective brain delivery. Additionally, EVs have effective endocytosis pathways that produce a higher rate of internalization than synthetic nanoparticles, making them more suitable for delivering therapeutic agents that act on intracellular compartments or organelles. These qualities collectively make EVs an improved type of drug delivery vehicle.

The goal of this project is to develop a new, effective therapy using state-of-the-art M/MD-targeted nanotherapeutics and hMSC-EVs for better combating ICH. While both components have been demonstrated previously, the proposed IL-10 carrying, M/MΦ-targeted hybrid EVs is a conceptually new design (FIG. 2) that has not been explored previously. Such a new therapy is more effective because it fully harnesses the immunomodulatory effects of both MSC-EVs and IL-10 (many possible other drugs as well), offering a significantly boosted treatment efficacy than any single component alone—a truly multivalent (nano-) therapeutic.

No previous systems have employed imaging to track or optimize the delivery of EVs. Such a multimodal imaging capability that is integrated in the new EV therapy provides unprecedented advantages compared to previous studies. Additionally, MRI and PET imaging are translated to large animal studies (exemplified by previous non-human primate studies) and to human trials as well.

Because the poor outcome of ICH is largely attributed to the activation and phenotypic polarization of microglia and macrophages (M/MΦ), a significantly improved outcome is achieved by a hybrid nanotherapeutic system composed of BBB-penetrating, M/MD-modulating hMSC-EVs and drug-carrying, M/MΦ-targeted liposomes (FIG. 1). The optimization in this disclosure is guided by multimodal imaging, including a novel MRI tracking method discussed in WO 2020/186253, herein incorporated by reference, as well as positron emission tomography (PET), fluorescence imaging (FI) and bioluminescence imaging (BLI). The presently disclosed imaging systems and methods have greatly improved tracking, guiding, and optimizing capabilities.

### Aim 1: Constructing, Characterizing, and Optimizing Hybrid EVs

Hybrid EVs are constructed from hMSC-EVs and PSL-IL10 using a PEG-mediated fusion method, and particle properties, cargo quantities, secretome, targeting ability, biological effects on M/MΦ, intracerebral delivery efficiency (both intranasal (i.n.) and intravenous (i.v.) administration) are characterized. Optimization is attained by adjusting the liposome formulation, EV/liposome ratios, and fusion conditions.

### Aim 2: Evaluating and Optimizing Hybrid EVs Treatment on Acute ICH

The effectiveness of hybrid EVs to promote phagocytosis and regulate inflammation in the acute phase of ICH (by day 7 post-ICH) is evaluated. Treatment outcomes are evaluated by lesion volume, neuroinflammation, hemoglobin content, and expressions of phagocytotic genes in M/MD. The efficacy of different treatment schedule/regimens is investigated to reach high treatment performance. Upon the completion of the proposed study, hybrid EVs are developed which are tested vigorously in large animal models and in clinical trials.

### Aim 3: To Improve the Efficacy of eEV-IL10 in ICH Mice

The therapeutic effect of engineered EVs loaded with IL-10 (eEV-IL10), in comparison with SC-EVs, PSL-IL10, or IL-10 alone are evaluated in a mouse ICH model. At the same time, because treatment efficacy is affected by the frequency, dose of the administration for a given type of treatment, a comprehensive evaluation of these factors is performed, which not only is beneficial for reaching a high treatment performance but also provides insight for future clinical translation and optimization in patient studies.

The disclosed hybrid EVs are easily tailored to the treatment of ischemic stroke and many other neuroinflammation-related CNS disorders.

The term “subject” as used herein refers to any individual or patient to which the disclosed methods are performed or from whom a biological material (e.g., sperm, a cell, or a biofluid) is obtained. Generally, the subject is human, although as will be appreciated by those in the art, the subject may be a non-human animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

In one embodiment, the present disclosure relates to a stem cell derived extracellular vesicle (SC-EV); and a liposome including a therapeutic agent. In one aspect, the liposome includes at least one of phosphatidylcholine or phosphatidylserine. In one aspect, the liposome is a phosphatidylserine (PS) liposome. In one aspect, the stem cell is a mesenchymal stem cell (MSC), a hematopoietic stem cell (HSC), an induced pluripotent stem cell (iPSC), an adipose tissue derived stem cell, or a neural stem cell (NSC). In one aspect, the therapeutic agent is a cytokine, an immunomodulatory agent, an antioxidant, an miRNA, or an antibody. In one aspect, the cytokine is interleukin-1 (IL-1), interleukin-2 (IL-2), interleukin-6 (IL-6), interleukin-10 (IL-10), interleukin-12 (IL-12), interleukin-15 (IL-15), interleukin-18 (IL-18), tumor necrosis factors (TNF), interferons (IFN), colony-stimulating factors (GM-CSF) or transforming growth factor-β (TGF-β). In one aspect, the immunomodulatory agent is a checkpoint inhibitor, a small molecule, or a chimeric antigen receptor. In one aspect, the antibody is adalimumab, infliximab, certolizumab, golimumab, or tocilizumab. In one aspect, the liposome further includes an imaging agent. In one aspect, the imaging agent is selected from superparamagnetic iron oxide (SPIO) nanoparticles, a lipid conjugated metal chelator, a radionucleotide, a fluorescent protein or a lipophilic fluorescence dye. In one aspect, the composition further includes a pharmaceutically acceptable carrier.

In one embodiment, the present disclosure relates to a method of generating a chimeric extracellular vesicle (cEV) including fusing a stem cell derived extracellular vesicle (SC-EV) and a liposome including a therapeutic agent, thereby generating a cEV. In one aspect, the liposome includes at least one of phosphatidylcholine or phosphatidylserine. In one aspect, the liposome is a phosphatidylserine (PS) liposome. In one aspect, the liposome is a unilamellar, multilamellar, oligolamellar, or multivesicular liposome. In one aspect, the stem cell is a mesenchymal stem cell (MSC), a hematopoietic stem cell (HSC), an induced pluripotent stem cell (iPSC), an adipose tissue derived stem cell, or a neural stem cell (NSC).

In one aspect, the therapeutic agent is a cytokine, an immunomodulatory agent, an antioxidant, an miRNA, or an antibody. In one aspect, the cytokine is interleukin-1 (IL-1), interleukin-2 (IL-2), interleukin-6 (IL-6), interleukin-10 (IL-10), interleukin-12 (IL-12), interleukin-15 (IL-15), interleukin-18 (IL-18), tumor necrosis factors (TNF), interferons (IFN), colony-stimulating factors (GM-CSF) or transforming growth factor-β (TGF-β). In one aspect, the immunomodulatory agent is a checkpoint inhibitor, a small molecule, or a chimeric antigen receptor. In one aspect, the checkpoint inhibitor is pembrolizumab, nivolumab, cemiplimab, atezolizumab, avelumab, durvalumab, ipilimumab, tremelimumab, or relatlimab. In one aspect, the antibody is adalimumab, infliximab, certolizumab, golimumab, or tocilizumab. In one aspect, the liposome further includes an imaging agent. In one aspect, the imaging agent is selected from superparamagnetic iron oxide (SPIO) nanoparticles, a lipid conjugated metal chelator, a radionucleotide, a fluorescent protein or a lipophilic fluorescence dye. In some aspects, the imaging agent is a microbubble, an iodine-based contrast agent, ¹⁸F, 2-deoxy-2-[fluorine-18]fluoro-D-glucose, lopamidol, lipiodol, ⁶⁸Ga, ¹¹C, ¹³N, or ¹⁵O.

In some embodiments, the miRNA is an anti-inflammatory miRNA. In one aspect, the miRNA is selected from: miRNA-122; miRNA-125; miRNA-199; miRNA-124a; miRNA-126; miRNA-98; Let7 miRNA family (for example, Let 7a, b, or c); miRNA-375; miRNA-141; miRNA-142; miRNA-148a/b; miRNA-143; miRNA-145; miRNA-194; miRNA-200c; miRNA-203a; miRNA-205; miRNA-1; miRNA-133a; miRNA-206; miRNA-34a; miRNA-192 miRNA-194; miRNA-204; miRNA-215; miRNA-30 family (for example, miRNA-30 a, b, or c); miRNA-877; miRNA-4300; miRNA-4720; and/or miRNA-6761.

In one embodiment, the techniques described herein relate to a method of treating inflammation including administering the composition described herein to a subject in need thereof. In one aspect, the inflammation is neuroinflammation. In one aspect, the subject has an intracerebral hemorrhage (ICH). In one aspect, the subject has an arterial hemorrhage, venous hemorrhage, or capillary hemorrhage. In another aspect, the subject has an epidural hematoma, subdural hematoma, subarachnoid hemorrhage, intracerebral hemorrhage, or intraventricular hemorrhage. In one aspect, the neuroinflammation is from an injury, infection, toxin exposure, neurodegenerative disease, or aging.

In one aspect, the administering is systemic, intranasal, intrathecal, intraventricular or intracerebral. In one aspect, the administering is intravenous, subcutaneous, or intramuscular.

In one embodiment, the present disclosure relates to a method of tracking or guiding cEV-based therapy including administering a composition described herein to a subject and detecting the cEV after administration. In one aspect, the tracking is by magnetic particle imaging (MPI), positron emission tomography (PET), fluorescence imaging (FI) or bioluminescence imaging (BLI). In one aspect, the tracking provides information for optimization of the cEV-based therapy.

In one embodiment, the dose of cEV is from about 1.0×10⁸ to 9×10¹⁰ particles per kg, about 1.0×10⁸ to 9.0×10⁸ particles per kg, about 1.0×10⁹ to 9.0×10⁹ particles per kg, about 1.0×10¹⁰ to 9.0×10¹⁰ particles per kg, about 1.0×10⁸ to 5×10⁸ particles per kg, about 6.0×10⁸ to 9×10⁸ particles per kg, about 1.0×10⁹ to 5×10⁹ particles per kg, about 6.0×10⁹ to 9×10⁹ particles per kg, about 1.0×10¹⁰ to 5×10¹⁰ particles per kg, or about 6.0×10¹⁰ to ×10×10¹⁰ particles per kg. In one aspect, the dose of cEV is about 10 μg to 100 μg cEV protein per kg, about 10 μg to 50 μg cEV protein per kg, about 20 μg to 40 μg cEV protein per kg, about 50 μg to 100 μg cEV protein per kg, about 80 μg to 100 μg cEV protein per kg, or about 40 μg to 60 μg cEV protein per kg. In one aspect, the dose is administered once, twice, three or four times a day. In one aspect, the dose is administered daily. In one aspect, the dose is administered once a day for 7-14 days. In one aspect, the dose is administered once a day for 1-7 days. In one aspect, the dose is administered once and not administered again. In one aspect, the dose is administered once a week, twice a week, or three times a week. In one aspect, the dose is administered for 1-14 days. In one aspect, the dose is administered for more than 14 days.

Presented below are examples of chimeric extracellular vesicles contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Construction of Hybrid EV

The PEG method was chosen because this method is gentle and efficient, allowing production of liposome-EV hybrids with low leakage of natural cargoes or loss of lipid bilayer integrity of EVs. This method also allows design of hybrid EVs with tunable composition and properties. In particular, hybrid EVs were developed by fusing EVs from various cells with PSL-IL10 at the molar ratio of 1:1 for 2 h at 37° C. using PEG (MW=8000, 20% w/v). These protocols are used in the present disclosure.

### Integrated Multimodal Imaging Capability for Tracking, Guiding, and Optimizing.

As illustrated in FIG. 2, hybrid EVs are formed by fusing hMSC and phosphatidylserine (PS)-containing liposomes (PSL-IL10). Liposomes are generated to carry not only therapeutic IL-10 but imaging agents ad hoc. Liposomes are encapsulated with superparamagnetic iron oxide (SPIO) nanoparticles (for MRI), lipid-conjugated metal chelator (i.e., Cu-DOTA for PET imaging), and fluorescence dyes (i.e., IRdye800cw or DiR for fluorescence imaging). FI and BLI are employed for not only quickly screening but also validating MRI and PET findings. Each of these imaging tools has strengths, allowing manipulation of the pharmacologic properties, including biodistribution, of hybrid EVs. For example, in this project, MRI is employed to assess the brain-delivery and subtle (intra-brain) distribution of the administered hybrid EVs in ICH mice, and PET is employed for whole-body bio-distribution and quantification of the hybrid EVs at the same time. A cutting-edge new imaging technology is also employed, MPI, which specifically detects SPIO tracers. MPI has desirable advantages, including zero-background (hot-spot) signals and excellent quantitative ability. For example, in this project, fluorescence imaging and BLI are employed for whole-body biodistribution research. MRI is used to assess the brain-delivery and subtle (intra-brain) distribution of the administered hybrid EVs in ICH mice and MPI or PET for whole-body bio-distribution and quantification of them at the same time. The combination of EV therapy and multimodal imaging capability offers unprecedented advantages for theranostics of ICH.

The construction hybrid EV showed encouraging therapeutic effects in ICH: PSL, PSL-IL10, EV, or hybrid EV (PSL-EV-IL10) were administered intravenously (i.v.) to ICH-subjected mice at 30 min after surgery and the injury volume was measured on day 3. Intravenous injected hybrid EVs suppressed the lesion volume (FIG. 4A), rescued the neuron viability (FIG. 4B), and increased mouse survival percentage compared to PSL-IL10 (day 3, PSL-IL10 vs. PSL-EV-IL10: 85.6% vs. 100%). This demonstrated the effectiveness of intranasally (i.n.) injected hybrid EVs in ICH mice. These results indicated stronger neuroprotection by hybrid EVs (FIG. 4) than that of PSL-IL10.

### Imaging Methods for Detecting Hybrid EVs

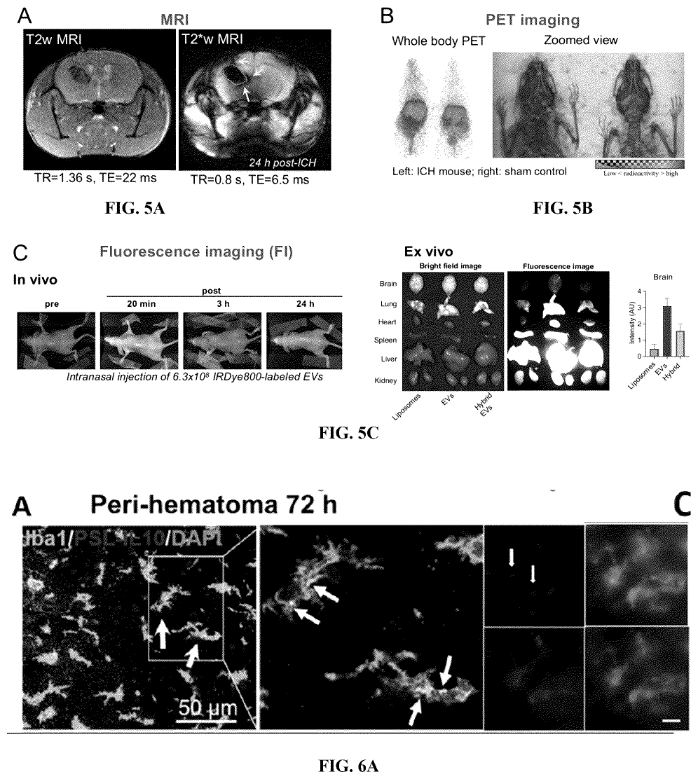

As shown in FIG. 5, multiple imaging methods were established for in vivo detecting and quantifying track EVs/hybrid EVs non-invasively. The imaging methods are complementary in terms of spatial resolution, sensitivity, quantification, and whole body coverage, providing an accurate and effective way to study the efficiency of intracranial delivery of hybrid EVs. MRI and PET are simultaneously conducted using a 7T PET/MRI small animal scanner.

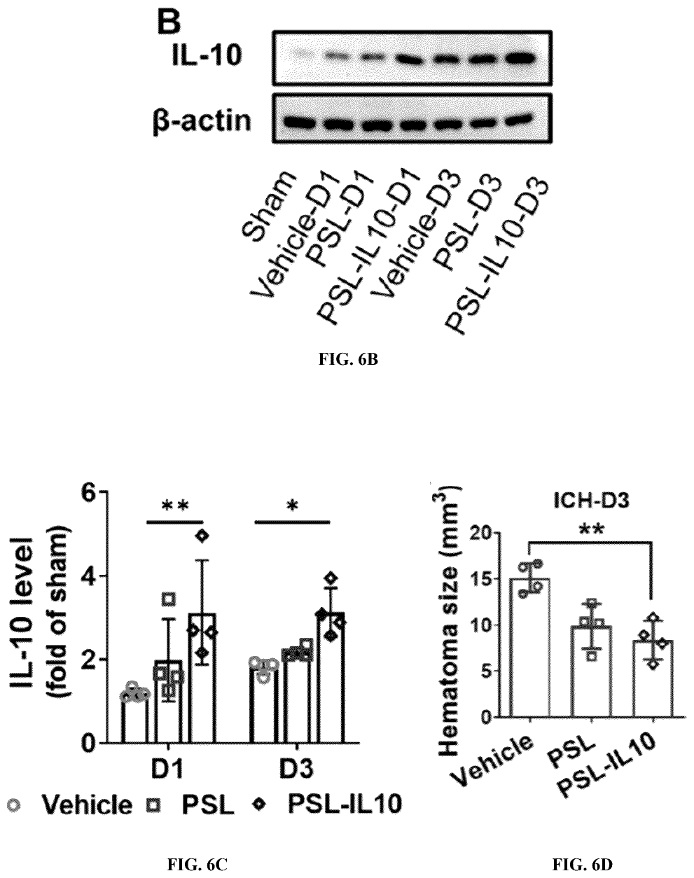

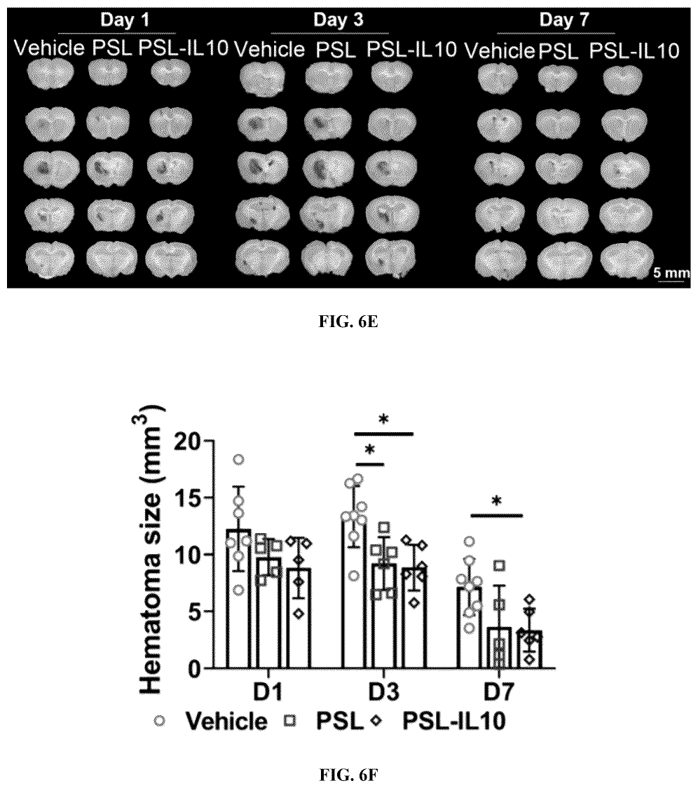

Mechanistic studies on the M/MDs targeting ability of PSL-IL10 revealed by in vivo studies: IL-10-loaded phosphatidylserine (PS)-modified liposomes (PSL-IL10) allow intracranial delivery of IL-10 in order to treat ICH. Phosphatidylserine (PS) is an “eat me” signal that is recognized by PS receptors mainly expressed in M/MΦs. The PS-modified liposomes (PSLs) were developed to mimic apoptotic cells that are selectively engulfed by M/MΦs in a noninflammatory manner, thus enhancing M/MΦ anti-inflammatory function. PSL-IL10 is intranasally administered (containing 0.3 μg IL-10) to mice at 30 min after ICH induction. Results showed that PSL-IL10 was selectively delivered to M/MΦs (FIG. 6A) rather than to neurons, astrocytes and endothelial cells (data not shown). Additionally, IL-10 levels increased in PSL-IL10-treated mice on day 1 and 3 post-ICH (FIG. 6B) and promoted hematoma resolution (FIG. 6C).

Stem cell-derived EVs have immunomodulation effects attributed to the cargo that they carry. The immunomodulatory effects of EVs derived from MSCs, iPSCs, and NSCs are harnessed by the described systems and methods. SC-EVs carry high-concentration intracellular antioxidant proteins peroxiredoxins (PRDX), which reduce oxidative stress and reduce apoptosis. Proteomics analyses reveal that iPSC-EVs have numerous significantly altered proteins compared to serum-derived EVs, with many of them associated with immune responses, wound healing, and hemostasis maintenance (Figure S12 in the publication Matsumoto, J. et al. “The transport mechanism of extracellular vesicles at the blood-brain barrier.” Current pharmaceutical design. 2017; 23(40):6206-6214, herein incorporated by reference), illustrating the immunomodulation effects of iPSC-EVs. In addition, miRNAs are an important category of bioactive molecules contributing to the functionality of EVs. In this context, anti-inflammatory levels of miRNAs in MSCs, iPSCs, and NSCs are substantially elevated. A recent study was conducted to compare the miRNA profiles between MSCs and iPSCs using RNA-seq, revealing distinctive immune-related miRNA profiles in human MSC- and iPSC-EVs. In addition to the described analyses, small RNA sequencing is conducted to analyze the secretome, focusing on miRNAs in the cEV product, which provides a mechanistic insight of the therapeutic effects of cEVs. Strengths and weaknesses in the rigor of the prior research: Consistent and standardized production of EVs remains an important technical consideration in EV research. The commonly used EV-isolating methods include (1) ultracentrifugation, (2) size exclusion, (3) immunological separation, and (4) polymer-based precipitation. Each method has its own advantages and disadvantages. Production procedure influences properties of EVs such as purity (i.e., protein-to-particle ratio), yield (number of particles per unit cell number), and quality (preservation of particle integrity). Different methods result in EV products with different sizes (and size distribution) and purity. The protocol herein described is consistently reproducible and produces EVs from cultured stem cells (Table 1). In one embodiment, an EV/liposome ratio (×4) of 1:1, 2:1, 4:1, 5:1, 6:1, 7:1, 8:1, 9:1, 10:1 is used. In another embodiment, an EV/liposome ratio (×4) of 1:10, 1:9, 1:8, 1:7, 1:6, 1:5, 1:4, 1:3, or 1:2 is used. In one embodiment, the EV/liposome ratio (×4) is between 10:1 to 1:10.

TABLE 1
describes cEV candidates assessed.

| | EV/liposome | |
|---|---|---|
| Stem Cells (x3) | ratio (x4) | IL-10 loading (x2) |
| Induced pluripotent stem cells | 1:1 | Intraluminal presenting |
| (iPSCs) | 2:1 | Extraluminal presenting |
| Mesenchymal stem cells | 4:1 | |
| (MSCs) | 8:1 | |
| Neuronal progenitor stem cells | | |
| (NPSCs) | | |

Commercial qEV columns (iZON) are used to streamline production of EVs with satisfactory yield and purity. Adhering to standard operating procedures ensures minimal batch-to-batch variations throughout the study. Rigorous characterization and quality control are carried out, including EV markers (transmembrane protein: CD63, CD81) and non-EV lipoprotein: ApoB100), according to the Minimal Information for Studies of EVs 2018 (MISEV2018) guidelines.

### Aim 1. Constructing, Characterizing, and Optimizing Hybrid EVs

The prepared hybrid EVs for targeting of M/MΦs and penetration of BBB is determined by the ratio of two types of membrane structures passed along from the original PSL and EVs. By fine-tuning the formulation of hybrid EVs, an optimized version of hybrid EVs is obtained, achieving the high delivery efficiency. Mechanistic studies are also conducted to determine the impact of fusion and labeling protocols on the functionalities of EVs in vitro, to preserve the therapeutic potential of SC-EVs and IL-10. EVs of different formulations are prepared according to Table 1 and FIG. 2, then the effects of the EVs on macrophages (MD) are assessed in vitro; for example, using organotypic hippocampal slice culture. The intracranial delivery efficiency of the EVs is assessed, including quantities and retention time. The sub-organ distribution of the EVs in the brain is assessed using NIR fluorescence imaging and MPI, or PET. The in vivo M/MΦ-targeting ability of the EVs is assessed using in vivo MRI and fluorescence microscopy. The highly effective EVs are tested in Aim 2.

Rationale: The prepared eEV-IL10 targets M/MΦs and penetrates BBB, determined by the ratio of two types of membrane structures passed along from the original PSL and SC-EVs. By fine-tuning the formulation of eEV-IL10, optimized versions of eEV-IL10 are obtained, to achieve high delivery efficiency for both brain penetration and M/MΦ targeting. Mechanistic studies are also conducted to investigate the impact of fusion and labeling protocols on the functionalities of EVs in vitro, to ensure the therapeutic potential of native SC-EVs and IL-10 are well preserved.

Design: various formulations of lead eEV-IL10 are prepared, and particle properties of cargo quantities and the secretome are characterized. M/MΦ targeting and brain delivery efficiency are evaluated using organotypic hippocampal slice culture (OHSC) and a tissue-engineered model of the human BBB. Immunoregulation and phagocytosis abilities of different formulations are assessed in vitro assays on both primary microglial cells and macrophage (MD) cell culture.

Data Relevant to Aim 1: Different SC-Derived EV Preparations have been Produced

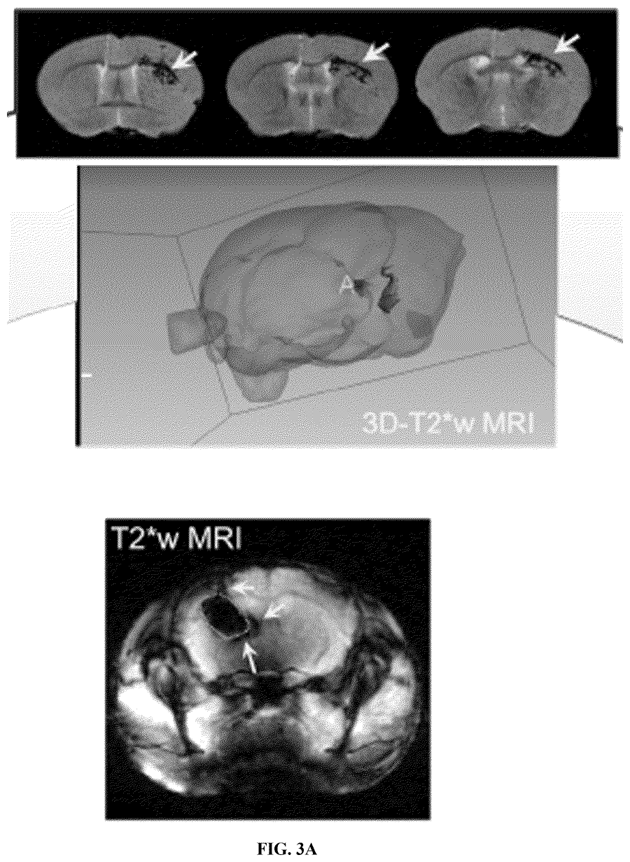

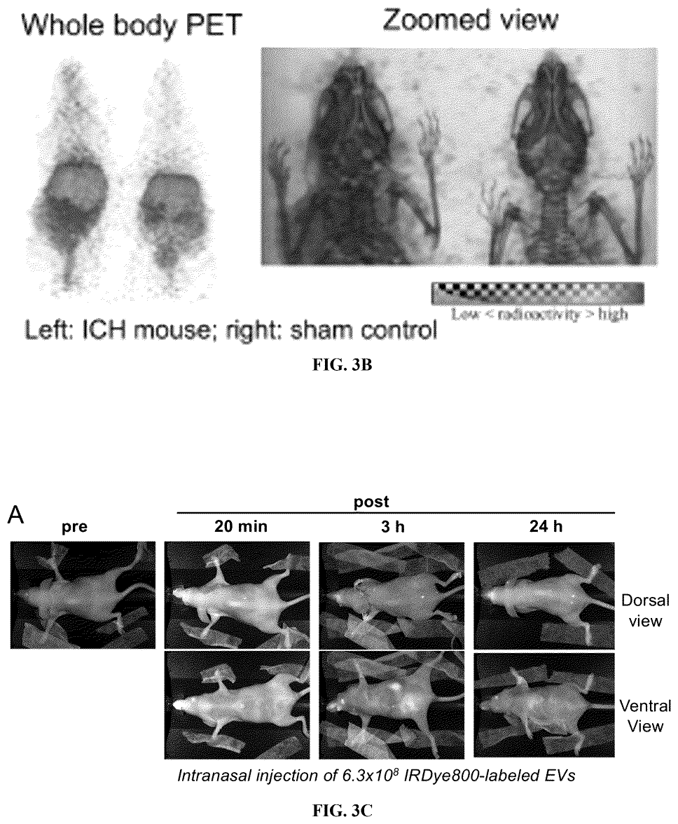

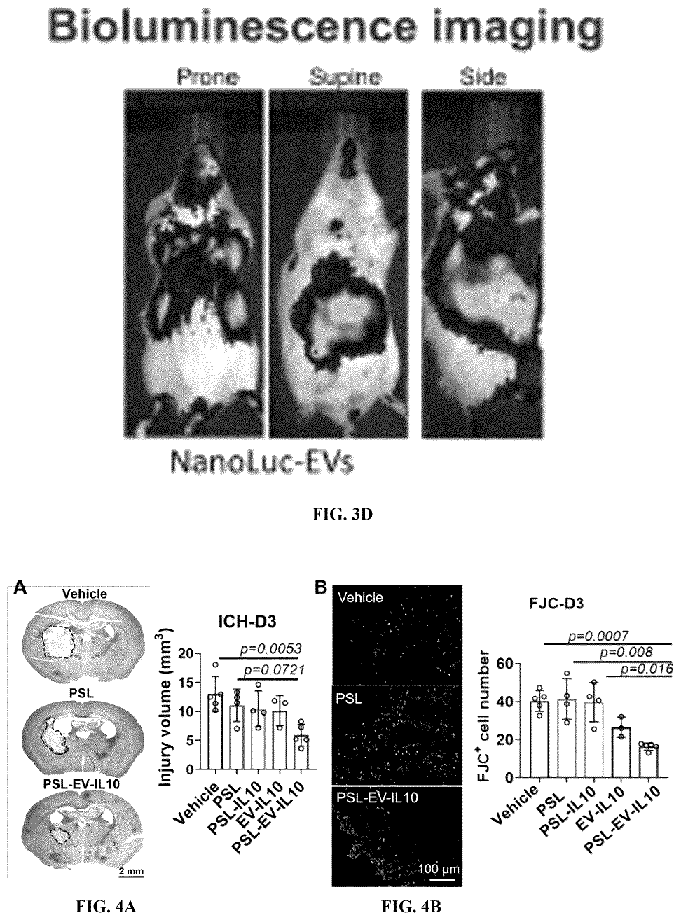

A prototype of cEV was prepared, and multimodal imaging of the constructed cEV was conducted in vivo (FIG. 3). The cEVs exerted protection against ICH injury (FIG. 4), providing strong evidence that cEVs are a promising drug candidate for ICH treatment. Therefore, in Aim 1, the construction, parameters, and characteristics of chimeric EVs are optimized. Formulations of cEVs are selected with the maximum M/MΦ targeting and brain delivery ability for the next step of treatment assessment. Based on PET (FIG. 3B or FIG. 3C) and in vivo fluorescence imaging (FIG. 7), intranasal injection is used to avoid non-specific uptake of vesicles/nanoparticles in the liver.

### Preparation of PSL-IL10 Liposomes

PS Liposomes, consisting of phosphocholine (PC) and phosphatidylserine (PS) lipid (molar ratio of 2.5:1), are prepared using the extrusion method with sizes homogenized using a final extraction through 0.1 m polycarbonate membranes. After being conjugated to palmitic acid-NHS, IL-10 (20 mg/mL) is incorporated into liposomes through hydrophobic interaction on the liposome surface or during extraction process intraluminally. Isolation of EVs: EVs are harvested from the serum-free culture medium of human MSC (Lonza) using ultrafiltration and commercial size exclusion columns (qEV columns, iZON).

### Methods

### Preparation of Stem Cells Derived EVs:

Human BC1 iPSCs (programmed from CD34+ hematopoietic progenitor cells of a healthy male donor) are cultured in 6-well plates coated with vitronectin (Gibco, Calsbad, CA) and maintained in Essential 8 (E8) medium (Gibco) supplemented with 10 μM Y-27632 dihydrochloride (Stem Cell Technologies, Vancouver, Canada) at 37° C. in a humidified atmosphere of 5% CO₂ and 95% air. The culture medium is changed daily after gently washing the cells with 10 mM PBS. Cells are passaged at 80%-90% confluence using TrypLE™ Express Enzyme (Gibco) supplemented with 10 μM Y-27632 dihydrochloride.

Human MSCs are acquired from Lonza (Catalog #: PT-2501) and cultured in MSC basal medium (MSCBM; Lonza).

Human NPCs are acquired from ATCC (Cat #ACS-5004), cultured in the plates coated with cell basement membrane (ATCC Cat #ACS-3035) using NPC Growth Medium (ATCC Cat #ACS-3003).

### EV Isolation:

Conditioned culture medium is centrifuged for 10 min at 300×g, followed by another 10 min at 2,000×g at 4° C. to remove cells and debris, and concentrated using an Amicon ultra-15 filter column and an Ultracel-100 membrane by centrifugation at 4,000×g for 20 min (MilliporeSigma, Billerica, MA, USA). Then, the concentrated EVs solution are purified by size exclusion chromatography (SEC) using qEV columns (iZON, Cambridge, MA, USA). Briefly, after rinsing the qEV columns with PBS, 0.5 mL fraction of the concentrated EVs are applied to the top of the columns and eluted with PBS each time. Three EV-rich fractions (7-9, 0.5 mL each) are pooled. The purified EVs are further concentrated using an Amicon column and final volume is adjusted so that the final concentration is ˜1×1011 EVs/mL.

Native EVs are labeled with DiR (ThermoFisher Scientific) according to manufacturer's instruction. In brief, 40 μL EVs (1.1×1011 EV/mL) are incubated with DiR at the final concentration of 1 μM for 15 min with gentle shaking, followed by eluting through a Sephadex G-50 column to remove free DiR dyes. At the same time, RhoB carried by liposomes provides additional fluorescence label on the resulted eEV-IL10, making them also RhoB labeled.

### EV Characterization:

The size and count of EVs are measured using a nanoparticle tracking analysis (NTA) instrument (Zetaview, Particle Metrix, Germany) using a 488-nm laser and ZetaView 8.04.02 software. To assess the size and shape, Transmission electron microscopy (TEM) is acquired using a Zeiss Libra 120 TEM operated at 120 KV and equipped with an Olympus Veleta camera (Olympus Soft Imaging Solutions GmbH, Münster, Germany). The loading of IL-10 is assessed by BCA assay. To assure the quality and purity of the EV products, rigorous characterization and quality control is conducted, including EV markers (transmembrane protein: CD63, CD81) and non-EV lipoprotein: ApoB100), according to the MISEV2018 guidelines.

Preparation of PSL-IL10 liposomes: PS Liposomes, including phosphocholine (PC) and phosphatidylserine (PS) lipid (molar ratio of 2.5:1), are prepared using the extrusion method with sizes homogenized using a final extraction through 0.1 m polycarbonate membranes. IL-10 is either added directly in the rehydrating solution (final conc=20 mg/mL), which results in intraluminal loading, or conjugated to palmitic acid-NHS to form IL-10 lipid, which is then incorporated into liposomes by post-insertion, which results in surface-presenting IL-10.

Preparation of cEVs:

Isolation of SC-EVs: EVs are harvested from serum-free culture medium of human MSC (Lonza, Cat #: PT-2501), NSPCs (ATCC #ACS-5004), and human BC1 iPSCs (programmed from CD34+ hematopoietic progenitor cells of a healthy male donor), and after being concentrated using ultrafiltration and size exclusion columns (qEV column, iZON). PSL-IL10 liposomes are prepared via the following methods: PS Liposomes, consisting of phosphocholine (PC) and phosphatidylserine (PS) lipid (molar ratio of 2.5:1), are prepared using the extrusion method with sizes homogenized using a final extraction through 0.1 m polycarbonate membranes. IL-10 is either added directly in the rehydrating solution (final conc=20 mg/mL), which results in intraluminal loading, or conjugated to palmitic acid-NHS to form IL-10 lipid, which is then incorporated into liposomes by post-insertion, which results in surface-presenting IL-10. Chimeric EVs (cEVs) are prepared via the following methods: cEVs of different formulations according to Table 1 are prepared by varying a) stem cell types, b) EV/liposome ratio, and c) IL-10 loading approaches. The particle concentrations of EVs and liposomes are determined using a nanoparticle tracking analysis (NTA, Zetaview).

### Fusion to Construct Hybrid EVs

The particle numbers of EVs and liposomes are counted using a nanoparticle tracking analysis (NTA) instrument (Zetaview). Mixtures of EV are prepared with the following methods: liposome with ratios of 1:1, 2:1, 4:1, and 8:1 are prepared, and PEG8000 is added to the mixture with a final concentration of 20% w/v. Fusion is stopped by removing PEG using ultrafiltration with 10K MWCO membranes. Hybrid EVs are measured for 1) size and size distribution using by DLS and TEM; and 2) EV markers by Western blots. EV labeling and purification: For MRI, SPIO-loaded liposomes are first prepared in the extrusion step by adding SPIO-His (0.2 mg/mL) with lipid solution, followed by purification using Ni-NTA. Hybrid EVs are then fused with SPIO-loaded liposomes. Iron content in the final product is measured using a spectrophotometric approach. For PET, liposomes are modified with DSPE-PEG2000-DOTA (synthesized by conjugating DSPE-PEG2000-NHS with DOTA-amine), followed by Cu chelation at pH 4.0 for 30 min. Free Cu is removed by ultrafiltration using a 3K MWCO filter. For Fluorescence imaging, hybrid EVs are labeled with IRDye800-labeled DSPE using the post-insertion method. For BLI, cells are transfected with NanoLuc luciferase, which allows use of BLI to quantify the organ distribution of EVs accurately.

One method of fusion follows: Fusion is induced by adding PEG8000 at a final concentration of 20% w/v and stopped by removing PEG using 10K MWCO filter membranes. Fusion efficiency measured by FRET-based lipid mixing assay: Liposomes are prepared to contain an equal molar ratio (1.5%) of the fluorescent NBD-PS and RhoB-PC. The fusion of liposomes with EVs results in increased distance between NBD-PS and RhoB-PE and thereby decreased RhoB(ex/em=535/583 nm) and increased NBD signal (ex/em=460/538 nm). Fluorescence signals are measured by a SpectraMax Plus 384 Microplate Reader and fusion efficiencies are calculated according to the published protocol.

### Labeling:

For Fluorescence imaging, cEVs are labeled with IRDye800-labeled DSPE using the post-insertion method. For MRI, SPIO-loaded liposomes are first prepared in the extrusion step by adding SPIO-His (0.2 mg/mL) with lipid solution, followed by purification using Ni-NTA. cEVs are then fused with SPIO-loaded liposomes. Iron content in the final product is measured using a spectrophotometric approach. For PET, liposomes are modified with DSPE-PEG2000-DOTA (synthesized by conjugating DSPE-PEG2000-NHS with DOTA-amine), followed by ⁶⁴Cu chelation at pH 4.0 for 30 min. Free Cu is removed by ultrafiltration using a 3K MWCO filter. For BLI, cells are transfected with NanoLuc luciferase, which allows us to use BLI to quantify the organ distribution of EVs accurately.

### Imaging

Different formulations of hybrid EVs are administered to mice through intravenous or intranasal injection (10⁹ particles per mouse, equivalent to 10 ng iron and 0.2 mCi64 Cu) at day 1 post-ICH mice, and a 7T PET/MRI scanner is used to conduct both PET and MRI at 30 min and 24 h after drug administration to assess the whole body and intracranial distribution respectively. Finally, FI and BLI are used to further verify the organ-level distribution of different particles ex vivo. Mass spectrometry (MS) is used to measure compare the biological molecules in the blood at different time points post hybrid-EVs injection.

In vitro characterization is conducted according to Table 1. One or several formulas of hybrid EVs are developed that effectively transverse BBB, accumulate in peri-hematoma and/or hematoma regions, selectively target M/MΦ, and/or promote desirable functionality changes in M/MΦ. In some aspects, alternative formulations of liposomes are developed to work effectively in all these aspects. Brain-targeted rabies viral glycoprotein (RVG) peptide is added in liposomes to improve BBB penetration. Mannosylated liposomes that specifically target M/MΦ by mannose-receptor are added to improve M/MΦ targeting. Throughout the study, quality control (QC) and validation studies are conducted to confirm the consistency, reproducibility, and robustness of the production process.

Compare the Therapeutic Potential of cEVs from Different SC Sources in Parallel.

The therapeutic potential of cEVs (eEV-IL10) formed from different SC-EVs is tested and compared for the first time. iPSCs are used because of their potential to replace the widely used MSCs with several advantages. iPSC-derived EVs are effective to repair ischemic myocardium, ameliorate skin aging, accelerate wound healing, and acute kidney injury. Compared to other SCs, iPSCs are generated from a single skin biopsy from the same patient, which makes it possible to generate individualized iPSC-EVs. This offers an exceptional translational promise through a continuous supply of autologous exosomes that is free of ethical concerns which exist with the use of hESCs30. Moreover, such individualized iPSC-EVs are unlikely to cause immune rejection. Compared to their parental cells, iPSC-EVs are much safer and non-tumorigenic, without compromising efficacy. Using iPSCs to produce EVs boosts the yield, standardization, and scalability of SC-EV production.

Characterization of cEVs

Immunoregulation effects of cEVs are evaluated using the selective uptake of cEVs among different populations of cells in OHSCs. In brief, brains are rapidly removed from 7 to 9-day old C57BL/6 mice and cut coronally into 350 μm thick slices with a Mcilwain Tissue Chopper. Hippocampal slices are placed on a 30-mm Millicell-CM insert membrane in six-well culture plates and cultured with 1 ml of culture medium. At 10-12 days in vitro, cultured slices are incubated with LPS (100 ng/ml), and 2×10⁸ vesicles/ml, fluorescently labeled cEVs (e.g., RhoB or DiR) of various formulations. At various time points after cEVs incubation, slices are fixed with 4% paraformaldehyde for immunostaining with cell markers (NeuN for Neuron, GFAP for astrocyte, Iba1 for microglia) and the ratio of fluorescence signal intensity of iba1+/RhoB+ to the total RhoB signal is used as a metric for M/MΦ targeting. Immunoregulation effects of cEVs are evaluated on bone marrow-derived macrophage culture using RNA-array, ELISA, and phagocytosis assay. Characterizations of cEVs also include a) size and size distribution assessed by Nano-FCM, NTA, and TEM, b) loading of IL-10 assessed by BCA assay, and c) EV markers characterized using Western blots.

### Imaging Experiments

MRI: All animal studies are performed on an 11.7T scanner with a T2*w FLASH sequence at different time points after the administration of 10⁹ EV, chimeric EV, or PSL-IL10 labeled with SPIOs (having the same iron amount) in 200 μL PBS. MPI: phantom or mice are scanned using a Magnetic Insight Momentum MPI scanner operating with a magnetic field gradient strength of 6×6T/m. 2D coronal projection images with a FOV of 6×10 cm are first acquired for localization, followed by 3D tomographic images with 55 projections. For in vivo quantification, volumetric ROIs are defined over the animal as well as laid over three “fiducials” markers. PET: EV, cEV, or PSL-IL10 modified with DSPE-PEG2000-DOTA, synthesized by conjugating DSPE-PEG2000-NHS with DOTA-amine, are used to chelate Cu⁶⁴ at pH 4.0 for 30 min. Free Cu⁶⁴ is removed by ultrafiltration using a 3K MWCO filter. Each mouse is injected with 0.2 mCi Cu⁶⁴ (equivalent to 10⁹ particles). FI (or BLI) is conducted using an IVIS® Spectrum CT system (Perkin Elmer).

Characterization of the BBB Permeability of eEV-IL10 Using a Tissue-Engineered Microvessel Model

To assess the ability of different eEV-IL10 to permeate BBB, a tissue-engineered BBB model is used. In brief, iPSC-derived brain microvascular endothelial cells (dhBMECs), mimicking the barrier function of the human BBB, are differentiated from BC1 iPSCs. After 11 days of differentiation, cells are characterized for expression of tight junction proteins, efflux pumps, and nutrient transporters as well as measurements of barrier function including transendothelial electrical resistance (TEER) or solute permeability, and only the cells with TEER>1500 Ωcm² are used. Then, dhBMECs are seeded onto 150 m diameter microchannels coated overnight with 50 μg mL⁻¹ human placental collagen IV (Cat #C5533, Sigma-Aldrich) and 25 μg/mL fibronectin from human plasma (Cat #F1141, Sigma-Aldrich) and maintained under a shear stress of 2 dyne/cm². Microvessels are perfused with fluorescent eEV-IL10 under live-cell imaging to determine the BBB permeability. Fluorescence signals in the receiving chamber and in the initial apical solution (C₀) are measured using the Synergy H4 plate reader (Biotek Instruments Inc., Winooski, VT) and are converted to particle concentrations using a priorly determined standard curve. The apparent permeability (Papp) is calculated as: Papp=dQ/dt×(V/A)×(1/C₀), where dQ/dt is the increase in particle concentration in the basolateral chamber, V is the volume of the basolateral chamber, A is the surface area of the transwell support, and C₀ is the initial particle concentration in the apical chamber. All permeability experiments are repeated using 3 different batches of dhBMECs. The presently described eEV-IL10 is a BBB-permeable, targeted delivery vector that facilitates IL-10 delivery, boosts in vivo efficacy, and advances its clinical use.

Immunoregulation effects of eEV-IL10 are evaluated using primary microglia cells and murine macrophage cell line. In brief, mouse pups at postnatal day 0-1 from C57BL/6 mice (Jackson Lab) are decapitated rapidly. The brains are removed and dissociated into single cells with papain. The primary glial cells are cultured with DMEM/F12 (#11330057, Thermo Fisher Scientific) with 10% fetal bovine serum (FBS) (#10438026, Thermo Fisher Scientific). At 14 days of culture in vitro, the primary glial cells are shaken at 200 rpm at 37° C. for 4 h. Then the primary microglia are collected from the supernatant and reseeded for 3 days before testing. The effect on macrophages is investigated by using RAW264.7 murine macrophage (ATCC, TIB-71) that are maintained in DMEM supplemented with 10% FBS. RAW264.7 are used at 80% confluency.

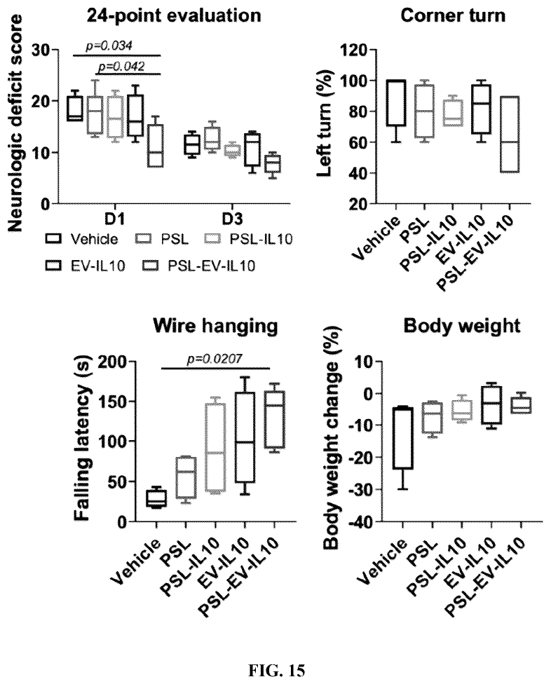

Quantitative Real-Time PCR (qRT-PCR, Exemplified by FIG. 14):

Primary microglia or RAW264.7 macrophage are treated with LPS (100 ng/mL) and IFN-γ (10 ng/mL) for 24 hours, and then changed to culture media containing IL-10 (10 ng/mL), native SC-EVs, or eEV-IL10 (3.2×10⁸ particles/mL, measured by NTA). At the end of incubation (48 hr), cells are collected to isolate the total RNA using 500 μL TRIzol RNA isolation reagents (Sigma) according to the manufacturer's instructions. The RNA concentration is determined using a Nanodrop 2000 spectrophotometer (Thermo Fisher Scientific). Complementary DNA (cDNA) is synthesized using SuperScript III First-Strand (Invitrogen), according to the manufacturer's instructions. Real-time polymerase chain reaction (RT-qPCR) is performed in SYBR Green reaction with a CFX96™ Real-Time System (BIO-RAD) using sequences of primers, i.e., IL1b (Mm00434228_m1), IL6 (Mm00446190_m1), IL4 (Mm00445259_m1), Arg1 (Mm00475988_m1), and GPADH (Mm99999915_g1). The endogenous reference gene, 18S ribosomal RNA (18S rRNA), is used as an internal control and normalization to calculate the fold changes in gene expression. All measurements include triplicate samples. FIG. 12 shows that eEV-IL10 (at the current formulation) has a similar IL-10-stimulation ability to PSL-IL10, however has a surprisingly weaker ability than iPSC-EVs.

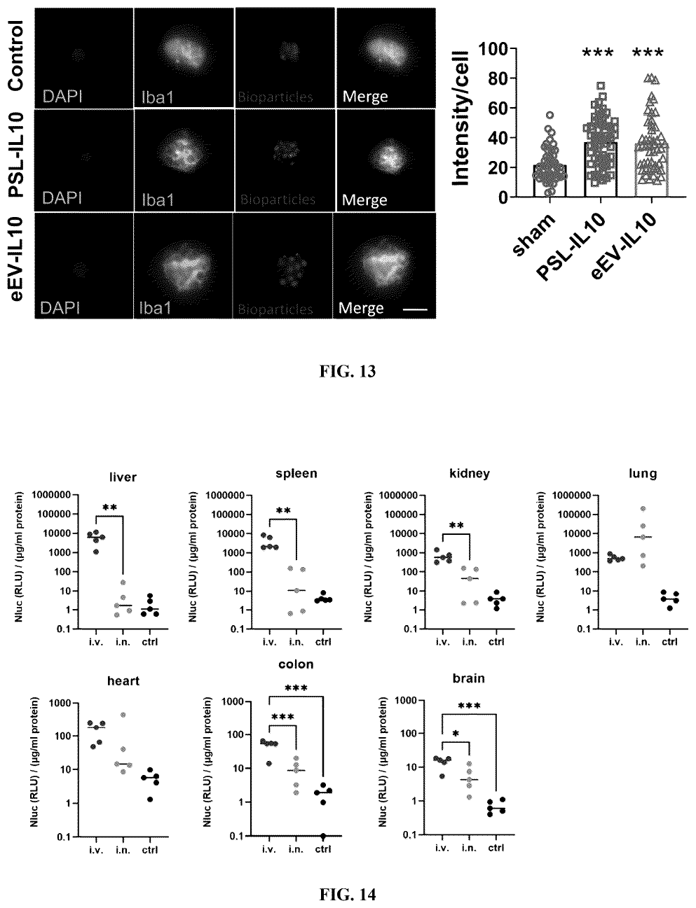

### Phagocytosis Assay (Exemplified by FIG. 13)

The primary cultured microglia or RAW264.7 are cultured in cell culture slide chamber (Thermo Scientific) at 10⁴ cells per well for 3 days and incubated with eEV-IL10 of various formulations (3.2×10⁸ particles/mL) for 24 h, then treated with pH-sensitive pHrodo® Red Zymosan Bioparticles (P35364, Thermo Fisher Scientific) according to the manufacturer's instructions. Due to the pH-sensitive nature of the fluorescence dye, the beads are only fluorescent (ex/em=560/585 nm) in endosomes following phagocytosis. Three hours later, cells are washed with PBS and fixed with 4% PFA. The images are taken with Leica microscope (DM6 FL, USA). The phagocytosis ability of cells are quantified by cellular fluorescent intensities. FIG. 13 shows that eEV-IL10 (at the current formulation) has a similar pro-phagocytosis ability to PSL-IL10.

### Dose-Titration Study

For the optimized 3-5 formulations, dose-response curves are determined with respect to the immunoregulation and pro-phagocytosis effects on both primary microglia and RAW264.7 using eEV-IL10 concentrations ranging from 1×10⁶ to 1×10⁹ particles/mL, with EC50 values estimated for each formulation.

### Expected Outcomes, Potential Pitfalls, and Alternative Strategies

One or several satisfactory cEVs formulations are developed that offer effective brain delivery to the perihematomal region and promote desired functionality changes in M/MΦ. The role of IL-10 receptors is also illustrated. In some aspects, alternative plans are incorporated: brain-targeted rabies viral glycoprotein (RVG) peptide are added to cEVs, and mannosylated liposomes are used to improve M/MΦ targeting.

### Construction of Hybrid EV

Three methods for EV-liposome fusion were compared, including polyethylene glycol (PEG), extrusion, and freeze/thaw. The PEG method was shown to gentle while efficient, allowing production of liposome-EV hybrids with lowered leakage of natural cargoes or loss of lipid bilayer integrity of EVs. This method also allows design of hybrid EVs with tunable composition and properties. A prototype of hybrid EVs was prepared by fusing EVs from various cells with PSL-IL10 at the molar ratio of 1:1 for 2 h at 37° C. using PEG (MW=8000, 20% w/v).

### Improving the Treatment Plan for Using Hybrid EVs to Treat Acute ICH

The efficacy of hybrid EVs over hMSC-EVs and PSL-IL10 alone in a mouse ICH model is evaluated. Because treatment efficacy is affected by the route, frequency, dose of the administration for a given type of treatment, a comprehensive evaluation of these factors is performed, which is not only beneficial for reaching an high treatment performance but also provides insight for clinical translation and optimization in patient studies. cEVs resulted in significantly reduced injury volume at 3 days after ICH (FIG. 4A) and decreased neural degeneration on day 3 compared to PSL-IL10 (FIG. 4B). With the improved brain delivery and optimized M/MΦ targeting, improved therapeutic outcomes are developed.

### Overall Strategy

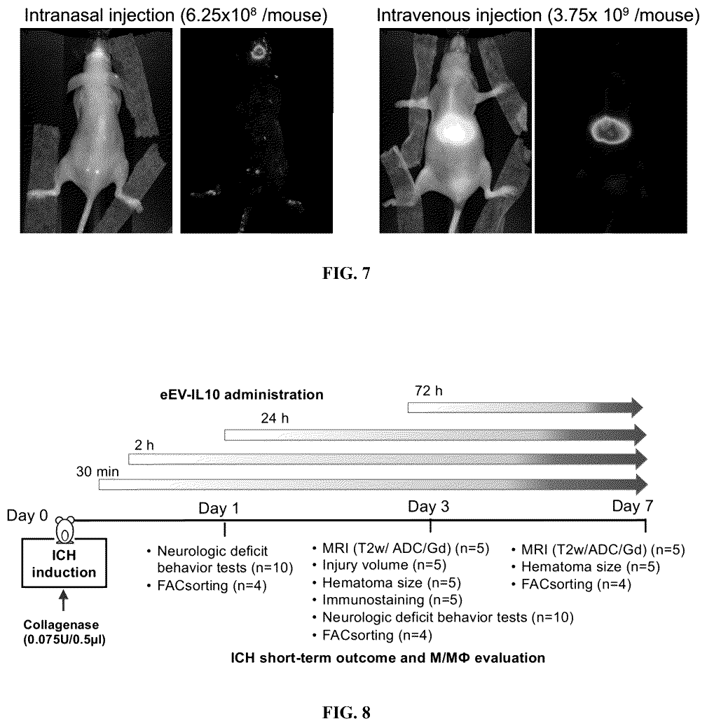

12-month-old male mice are subjected to the collagenase-induced ICH model (0.075 U/0.5 μl) to evaluate treatment outcomes. Sham-operated mice are injected with saline. All in vivo experiments are performed in accordance with the STAIR and RIGOR guidelines. Animals that die during surgery or before treatment are excluded from further studies. Treatment plan: mice are randomized in a blinded manner for treatment with PSL-IL10, (native and labeled) hMSC-EVs, (native and labeled) hybrid EVs, and vehicle (saline) controls. a) The time point of single injection or staring time point of multiple administration: 30 min, 2 hours, 24 hours, and 72 hours (post-ICH). b) Injection route and regimen (dose and frequency): intravenous and intranasal administration; single injection (3×10⁹ per mouse) vs 3 separated injections (10⁹ per injection every 24 hours). Sham-operated mice are injected with saline. The overall design is illustrated in FIG. 8.

### Evaluation of Outcomes

All outcomes are assessed by an investigator blinded to group identity. All results (negative and positive), a priori exclusion/inclusion criteria, and power/sample size calculations are recorded. A 24-point scoring system, wire hanging test, and corner turn test are used to evaluate neurologic deficits on days 1, 3, and 7 after ICH. Lesion volume is measured using T2-weighted (T2w) MRI, and the brain edema is evaluated with diffusion tensor imaging (DTI)/MRI on days 3 and 7 post-ICH. After MRI scanning, the mice brain is harvested and prepared for morphological assessment: a) using Luxol Fast blue staining to confirm and measure the lesion volume and brain edema; b) evaluate the neuronal death with FJC staining; c) hemoglobin content using a hemoglobin assay kit; d) address the immune regulation of hybrid EVs on M/MΦ's polarization. Flow cytometry is used to collect the M/MΦ on day 3 after ICH; microglia are identified as CD45intCD11b+ and macrophages are identified as CD45highCD11b+; then mRNA is extracted and levels of compounds associated with M/MΦ inflammation and phagocytosis are measured using RT-qPCR, including IL-1β, TNF-α, CD86, IL-4, IL-10, Ym1, Arg1, CD86, CD36, TREM2, mfge8, and others. Immunostaining is performed with Iba1 and CD68 antibodies, pro-inflammatory markers (CD16/32, iNOS, and CD86), and anti-inflammatory markers (Ym1, CD206, and Arg1). All histological images are analyzed with ImageJ software. Standard histological and biological assessments are outsourced to the core service centers at Johns Hopkins, which streamlines the workflow and saves effort.

### Methods

A 24-point scoring system, wire hanging test, and corner turn test are used to evaluate neurologic deficits on days 1, 3, and 7 after ICH. Brain injury volume, brain edema, and BBB integrity are assessed using T2w, DTI-MRI, and Gd-DCE MRI. Pharmacokinetic analysis on the BBB permeability (i.e. Ktrans) are analyzed using Gd-DCE data according to published protocols. After the final MRI scan, mouse brains are harvested and freshly cut coronal brain sections (1-mm thick) are used to measure the hematoma volume and Evans Blue measurement is used to evaluate the BBB integrity. Histological assessments are performed, including a) Luxol Fast blue staining is used to measure the lesion volume and brain edema, b) FJC staining is used to evaluate the neuronal death, and c) Iba1 and CD68 staining is used for M/MD. To determine the effects on hematoma resolution, ipsilateral hemispheres are homogenized to measure the hemoglobin content using a hemoglobin assay kit. Flow cytometry is used to collect the M/MΦ, with microglia identified as CD45int CD11b+ and macrophages as CD45high CD11b+, then levels of mRNA associated with M/MΦ function and phagocytosis are measured using qRT-PCR.

### Power Analysis and Statistics

Cohort sizes are determined using power analysis. Statistical comparisons among multiple groups are made by ANOVA (one-way, two-way, or repeated measures depending on the number of variables) followed by Bonferroni/Dunn post hoc correction. Differences between two groups are determined by two-tailed Student's t-test. If the data are not normally distributed, nonparametric tests are used. Statistical significance is set at p<0.05.

### Rigorous Experimental Design for Robust and Unbiased Results

12-month-old mice of both genders are used to evaluate treatment outcomes. All in vivo experiments are performed in accordance with the STAIR and RIGOR guidelines. Animals that die during surgery or before treatment are excluded from further studies. All measurements are conducted by an investigator blinded to group identity. All results (negative and positive), a priori exclusion/inclusion criteria, and power/sample size calculations are recorded. Using a 80% power for detecting differences in the neurologic deficits and lesion volume, in one aspect, power analysis determines the appropriate cohort size to be 8 mice per group, or 10 mice considering ˜85% survival in 12-month-old mice. The statistical comparisons among multiple groups (treatment vs. vehicle; behavior tests at different time points) are made by ANOVA (one-way, two-way, or repeated measures depending on the number of variables) followed by Bonferroni/Dunn post hoc correction. Differences between two groups are determined by a two-tailed Student's t-test. Covariance is analyzed by multiple regression models. For data not normally distributed, nonparametric tests are used. Statistical significance is set at p<0.05.

### Benchmark for Success

The presently disclosed hybrid EV-based therapy has a higher treatment efficacy than PSL-IL10 and EVs alone (significance P<0.05). Outcomes are quantitatively measured and compared. In one aspect, therapeutic efficacy reduces the lesion volume (assessed by MRI) by at least 50% on Day 3. In addition, brain delivery and M/MΦ targeting is correlated with therapeutic outcomes, which serves as the basis for later optimization, in which imaging is used as guidance. Rigorous quality control (QC) process is used, and optimized formulations are determined for future studies.

### Outcomes, Pitfalls and Strategies

Significantly improved outcomes are observed when the hybrid EVs treatment starts at 30 min and 2 hours post-ICH. In one aspect, no significant improvement is observed within 7 days in the 1 day or 3 days treatment groups, and the duration of treatment is expanded to up to 28 days until significantly outcomes (assessed by MRI) are observed.

### Example 2

### Enhanced M/MΦs-Targeting Achieved Using EV Engineering

Another scientific premise of the present disclosure is the enhanced effectiveness of engineered EVs in targeting M/MDs. The present results, as shown in FIG. 10, demonstrate a significantly improved M/MD-targeting ability of eEV-IL10 compared to PSL-IL10 in mouse brains with ICH. Quantified by the ratio of Iba1+/RhoB+ cells to the total Iba1+ cells, the selective uptake of eEV-IL10 in microglia was determined to be increased by 56%. This indicates the potential of engineered EVs to enhance M/MΦs-targeted delivery.

### Reproducible Protocol

A consistently reproducible protocol is used to produce EVs from cultured stem cells (FIG. 12). Commercial qEV columns (iZON) are used to streamline the production of EVs with satisfactory yield and purity. Adhering to standard operating procedures ensures low batch-to-batch variations throughout the production of cEVs. Rigorous characterization and quality control is conducted, including use of EV markers (transmembrane protein: CD63, CD81) and non-EV lipoprotein: ApoB100), according to the Minimal Information for Studies of EVs 2018 (MISEV2018) guidelines.

### Aim 2: To Assess the Pharmacokinetics and M/MΦs-Targeting in ICH Mice

Rationale: the time-dependent eEV-IL10 distribution is examined at both the organ and cellular levels. For each formulation, effects of injection routes are compared: a) direct intracranial administration, b) intranasal (i.n.) administration, and c) intravenous (i.v.) administration. Following the pharmacokinetic test, the in vivo efficacies are assessed, and correlation between the outcomes and local eEV-IL10 concentrations are determined, which are utilized to estimate the minimal effective dose, which is used to design the dose schedule.

Design: A panel of complementary imaging modalities is employed to assess the pharmacokinetics, brain uptake and distribution. Bioluminescence imaging (BLI) is used as the main modality for biodistribution assessment because BLI is less prone to the errors of loss (or transfer) of imaging labels from EVs in vivo than fluorescence imaging. The feasibility of using BLI to precisely determine biodistribution is shown, for example, in FIG. 14.

In vivo NIR fluorescence imaging and MPI are used to assess the pharmacokinetics. MRI is employed to assess the high-resolution intra-brain distribution. When needed, PET is used to provide (more accurate) pharmacokinetic assessments. Finally, histological assessments are performed after imaging, not only to validate imaging findings but to evaluate M/MΦs targeting and therapeutic effects.

### Methods

### Imaging Labeling:

For BLI, parental stem cells are transfected with NanoLuc, which allows us to use BLI to quantify the biodistribution of eEV-IL10 accurately. For fluorescence imaging, eEV-IL10 is labeled with IRDye800-labeled DSPE using the post-insertion method. For MRI, SPIO-loaded liposomes are first prepared in the extrusion step by adding 20 nm SPIO-His (0.2 mg/mL) with lipid solution, followed by purification using Ni-NTA. eEV-IL10 are then fused with SPIO-loaded liposomes. Iron content in the final product is measured using a spectrophotometric approach. For PET, liposomes are modified with DSPE-PEG2000-DOTA (synthesized by conjugating DSPE-PEG2000-NHS with DOTA-amine), followed by ⁶⁴Cu chelation at pH 4.0 for 30 min. Free ⁶⁴Cu is removed by ultrafiltration (3K MWCO).

### Ich Model:

Under anesthetization using 1%-3% isoflurane inhalation and ventilation with oxygen-enriched air (20% 02:80% air) by a nose cone, mice receive an intracranial injection of 0.5 μL of collagenase VII-S (#C2399, MilliporeSigma) at 0.15 U/μL (0.1 μL per minute) in the left striatum (0.6 mm anterior and 2.0 mm lateral to the bregma, and 3.0 mm in depth). During the operation, body temperature of the mice is maintained at 37.0±0.5° C. Sham-operated mice receive a saline injection at the same location and injection volume. All animals are included in the final analysis, except those who die or have a >21 out of 24 score on the neurologic deficit assessment within 24 hours of surgery.

Imaging experiments: Animals are randomly assigned to groups: 1) eEV-IL10 of different formulations, 2) PSL-IL10, 3) native EVs, and 4) saline control. Each group is further separated into three subgroups to receive intraventricular (i.v.), intranasal (i.n.), intravenous (i.v.) injection of solution containing 0.3 μg IL-10 equivalent of PSL-IL10, equivalent PSL, or 0.3 μg of IL-10, or saline, respectively. The injection is performed at 2 hours post-ICH. Imaging is performed at selected time points: 30 min, 1, 2, 3, 4, 6, 12 and 24 hours. After the final time point of in vivo imaging, mice are perfused with PBS via cardiac puncture. Organs are harvested and imaged freshly (fluorescence and BLI imaging) or fixed in PFA at 4° C. for 24 hours (MRI and MPI).

BLI (ex vivo, exemplified by FIG. 16): Freshly collected organs are imaged ex vivo in bioluminescence mode of an IVIS Spectrum (PerkinElmer) while immersed in NanoGlo substrate (diluted 1:50, Promega #N1110). Tissues are then homogenized in 1 ml N-PER (brain) or T-PER (other organs)+Complete Mini protease inhibitor cocktail tablet (Roche, Mannheim, Germany) in FastPrep Lysing Matrix D tubes on a FastPrep homogenizer. Homogenates are centrifuged for 5 min at 10,000×g at 4° C., and the supernatant is measured by NanoGlo and BCA assays (Pierce, Rockford, IL) for the amount of NanoLuc enzyme activity.

Fluorescence imaging (in vivo and ex vivo, exemplified by FIG. 8): Both in vivo and ex vivo imaging are conducted using a Pearl Trilogy small animal imaging system (LI-COR Biosciences). The fluorescence intensity is measured with the 800 nm channel and analyzed with Image Studio Ver. 5.0 software (LI-COR Biosciences) or ImageJ.

MPI (in vivo and ex vivo, exemplified by FIG. 3): MPI is conducted using a Magnetic Insight Momentum MPI scanner operating with a magnetic field gradient strength of 6×6T/m. 2D coronal projection images with a FOV of 6×10 cm are first acquired for localization, followed by 3D tomographic images with 55 projections. For in vivo quantification, volumetric ROIs are defined over the animal as well as laid over three “fiducials” markers.

MRI (in vivo and ex vivo, exemplified by FIG. 3): In vivo MRI is conducted, pre, 30 min, 2 hours and 24 hours after the injection of eEV-IL10 (labeled with 20 nm SPIO) using an 11.7T Biospec (Bruker) horizontal bore scanner equipped with a mouse brain surface array RF coil (receiver) and a 72 mm volume coil (transmitter). T2* maps are acquired using a multiple gradient echo (MGE) pulse sequence with TR=800 ms and eight TE times ranging from 3 to 18 ms. Other acquisition parameters are matrix size=256×128 and resolution=167×280 mm². Ex vivo high-resolution MRI is performed using a vertical bore 11.7T Bruker scanner equipped with a 15 mm birdcage transmit/receive volume coil. A 3D FLASH sequence is used with TE=6 ms, TR=150 ms, FOV=12×9×6 mm, isotropic resolution=0.04 mm3, averages=5, and flip angle=15. The total scan time is 6 h and 43 m. 3D-slicer is used to quantify the areas of hypointense signal and visualize the 3D distribution of different particles.

PET (in vivo, exemplified by FIG. 3): PET imaging is only performed on selected groups to confirm the pharmacokinetic results. Each mouse is injected with 0.2 mCi Cu⁶⁴ (equivalent to 10⁹ particles). Images are acquired with a 7T/30 Bruker Biospec MRI scanner (Bruker BioSpin) with a three-ring Bruker Si 198 PET insert. Simultaneous whole-body MRI and 10-minute static PET scans are acquired using a 72-mm PET-optimized Tx/Rx radiofrequency coil (Bruker BioSpin) centered inside the PET detector.

Histological assessment is performed to 1) assess the initial therapeutic effects and 2) validate the imaging findings about the intra-organ and intracellular distribution of eEV-IL10.

Immunostaining: Brain tissues in 25-pm-thick coronal sections are processed and stained. Different antibodies are applied, including anti-glial fibrillary acidic protein (GFAP, 1:500, #13-0300, Life Technologies), anti-CD36 (1:100, #MA5-14112, Invitrogen), anti-CD68 (1:200, #MCA1957, Bio-Rad Laboratories, Hercules, CA, USA) and anti-ionized calcium binding adaptor molecule 1 (Iba1) antibody (1:500, #019-19741, Wako, Osaka, Japan) and incubated overnight at 4° C. After appropriate secondary antibody incubation, the nuclei are labeled by DAPI (H-1200, Vector Laboratories, Burlingame, CA, USA). Sections are examined with fluorescence microscopes (Leica DM6, FL, USA or EVOS FL auto imaging, Thermo Fisher Scientific, Frederick, MD) or a confocal microscope (Leica SP8). Prussian blue staining is conducted to detect SPIO-labeled eEV-IL10.

Microglia/macrophage morphology analysis: The morphology of perihematomal Iba1-positive cells are analyzed using Neurolucida software (MBF Bioscience). Three fields per section with a 200× magnification (˜120 cells) in each image are included to quantify cell soma size, dendrite number, and length, number of nodes and ends of Iba1+ cells, and the complexity of the cells.

### Magnetic-Activated Cell Sorting (MACS):

To determine the immune regulation effect and provide mechanistic insights of the therapeutic effects, M/MΦs are isolated from the mouse brain after ICH without and with eEV-IL10 injection. In brief, on day 1 post-ICH (after final imaging study), a 4 mm-thick section of the ipsilateral brain hemisphere is collected from each randomly selected mouse in cold Hanks' buffered salt solution (HBSS; without Ca²⁺/Mg²⁺). Tissue dissociation is conducted using the GentleMACS™ Dissociator and Neural Tissue Dissociation kit (P) (#130-092-628, Miltenyi Biotec, Bergisch Gladbach, Germany). The single cell suspension is harvested after the myelin is removed using myelin removal beads (#130-096-731, Miltenyi Biotec), and M/MΦs are magnetically separated with CD11b MicroBeads (#130-049-601, Miltenyi Biotec) and LS columns. The sorted cells are then applied for ex vivo phagocytosis assay or mRNA extraction.

Ex vivo M/MΦ phagocytosis assay: The sorted CD11b+ cells are seeded on a poly-1-lysine-coated 96-well plate at 10⁴ cells per well and cultured in DMEM/F-12 (#11330057, Thermo Fisher Scientific) with 10% FBS (#10438026, Thermo Fisher Scientific), 20 ng/μL M-CSF (#315-02, PeproTech) and 100 U/mL penicillin-streptomycin for 16 hours. The cells are incubated with pH-sensitive pHrodo® Red Zymosan Bioparticles (P35364, Thermo Fisher Scientific) for 3 h. Then, the cells are washed with PBS and fixed with 4% PFA, followed by immunostaining with Iba1 primary antibody and Alexa Fluor 488 conjugated secondary antibody. The images are taken with EVOS FL auto imaging (Thermo Fisher Scientific). qRT-PCR is performed as described.

### Aim 3: To Improve the Efficacy of eEV-IL10 in ICH Mice

The therapeutic effect of eEV-IL10, in comparison with SC-EVs, PSL-IL10, or IL-10 alone are evaluated in a mouse ICH model. At the same time, because treatment efficacy is affected by the frequency, dose of the administration for a given type of treatment, a comprehensive evaluation of these factors is performed, which not only is beneficial for reaching a high treatment performance but also provides insight for future clinical translation and optimization in patient studies. Experiments show that eEV-IL10 resulted in significantly reduced injury volume at 3 days after ICH (FIG. 4A) and decreased neural degeneration on day 3 compared to PSL-IL10 (FIG. 4B). With the improved brain delivery and optimized M/MΦ targeting, further improved therapeutic outcomes are observed. Moreover, the neurologic deficit behavior test results suggest the desired efficacy of eEV-IL10 even though the sample size is small in this study (FIG. 17).

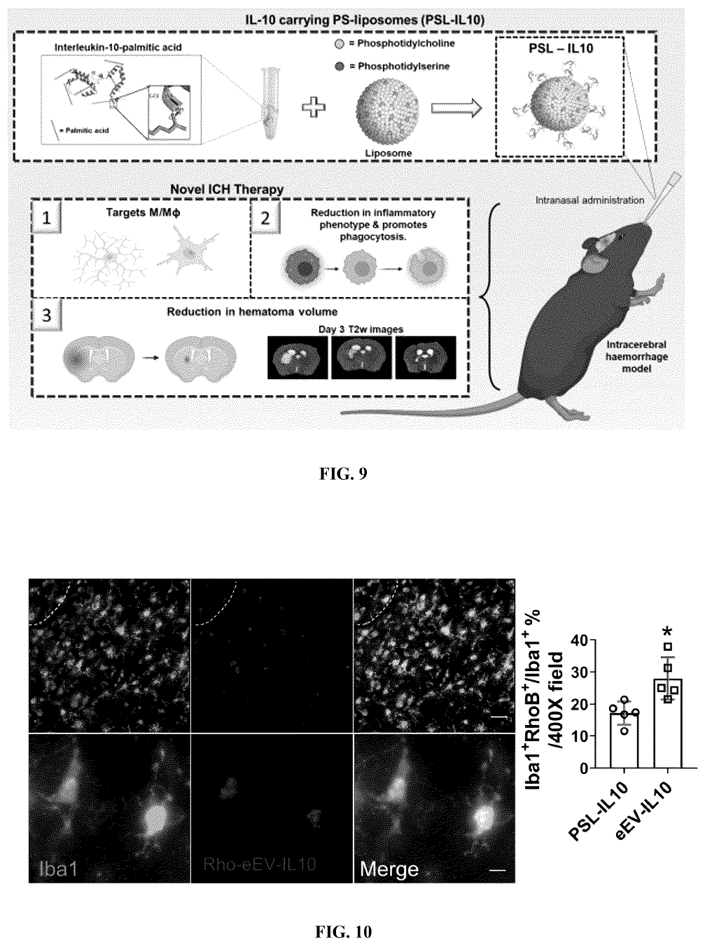

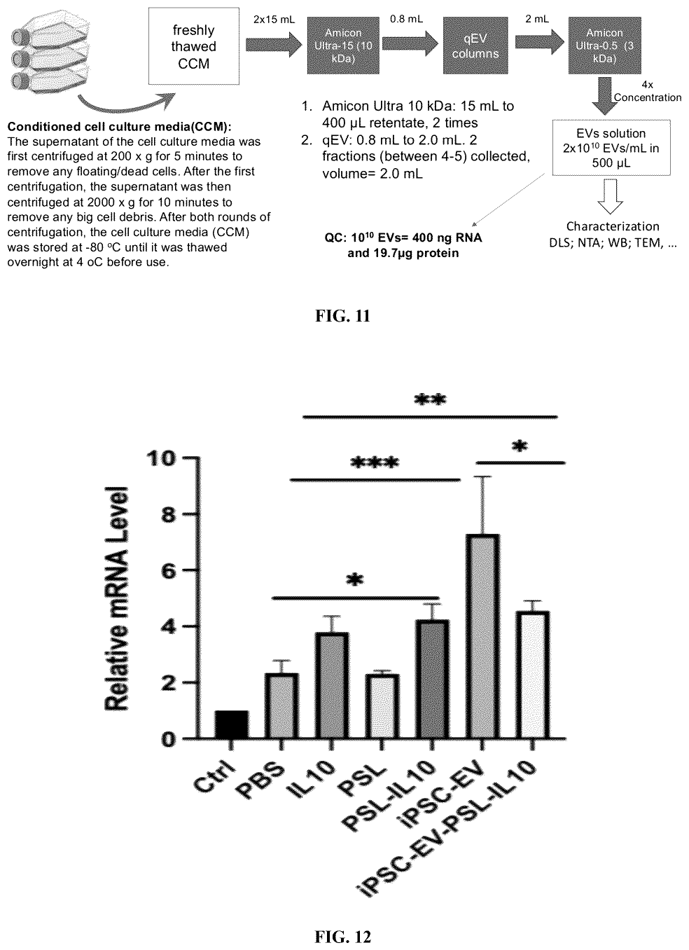

Design: The overall design is illustrated in FIG. 9. Mice are randomized in a blinded manner for treatment with IL-10 alone, PSL-IL10, SC-EVs, eEV-IL10, and vehicle (saline) controls. Using the injection route determined in Aim 2, ICH mice receive a) a single injection at 0.5 h, 2 h, 24 h, or 72 h (post-ICH) at the dose of 3×MED, where MED=minimum effective dose determined in Aim2, or b) 3 separated injections (q1d×3) at MED, started at 0.5, 2, 24, or 72 h (post-ICH). During the course of study, all animals are monitored for body weight change daily within the first week of ICH.

Lesion volume and hematoma size analysis: Three days after ICH, brain lesion volume is evaluated by staining 25-pm-thick brain sections with Luxol fast blue (FB) and cresyl violet (CV). The hematoma size at 1, 3, or 7 days after ICH is measured by scanning the freshly cut hemorrhagic brain sections (thickness=1 mm).

Brain water content: As a surrogate biomarker for brain edema, the brain water content is measured using a wet-dry weight method. At 3 days after ICH, mice are euthanized by deep anesthesia. The brains are removed and divided into ipsilateral or contralateral cortex, striatum, and cerebellum to obtain the wet weight. Then, the dry weight is collected after drying tissues at 100° C. for 24 hours. The brain water content is calculated as [(wet weight-dry weight)/wet weight]×100%.

MRI: A horizontal 11.7 Tesla MR scanner (Bruker Biospin, Billerica, MA, USA) equipped with triple-axis gradient (maximum gradient strength=74 Gauss/cm) using a volume excitation coil and a 4-channel phased array mouse head receive-only coil is applied to perform in vivo MRI experiments. Brain injury volume, brain edema, and BBB integrity are assessed using T2w, DTI-MRI, and Gd-DCE MRI, respectively. Pharmacokinetic analysis on the BBB permeability (i.e., Ktrans) is analyzed using Gd-DCE data. Hematoma volume is measured on days 3, 7, and 14, residual lesion volume on days 3, 7, 14 and 28, and volume of the contralateral hemisphere in the T2w images using a 3D slicer.

Western blot: Samples with an equal amount of total protein are loaded into and separated by 4-20% SDS-PAGE gel electrophoresis and transferred onto PVDF membrane. After blocking, the antibodies to the following proteins are applied for membrane incubation overnight: IL-10 (1:1000, ab9969, Abcam), STAT3 (1:1000, #4904, Cell Signaling Technology), phosphor-STAT3 (1:1000, #9145, Cell Signaling Technology), CD36 (1:1000, #MA5-14112, Invitrogen), b-actin (1:2000, sc-47778, Santa Cruz). Then after incubation with appropriate horseradish peroxidase-conjugated secondary antibodies, the membranes are immersed in enhanced chemiluminescence solution to detect bands under an ImageQuant ECL imager (GE Healthcare) or iBright™ CL1000 imaging system (Themofisher Scientific). Bands are further analyzed by ImageJ software. All data are normalized to the corresponding loading control and expressed as fold change to the sham group.

Systemic immune reactions and adverse effects: Antibody and cytokine measurements are performed. In brief, total IgG levels in mouse plasma are measured using a mouse IgG ELISA kit (Abcam, ab151276). Plasma samples, collected 24 h after EV administrations, are diluted 70,000× in sample diluent, and IgG levels are measured following the manufacturer's instructions. The plate is measured on a BioRad plate reader at 450 nm. EV-specific antibody levels are measured by ELISA. Cytokine levels are measured using the LEGENDplex mouse Inflammation Panel in filter plates (BioLegend). Plasma samples are diluted 1:4 in assay buffer and cytokine levels are measured following manufacturer's instructions using a BD LSR Fortessa flow cytometer. Moreover, the bodyweight in all the groups, especially in the sham control group, are used as a metric to evaluate the drug toxicity of eEV-IL10, with the dose producing >20% body weight loss defined as the maximum tolerated dose (MTD).

Statistics and Correlation analysis: Data are presented as means±SD or box-and-whisker plots. Differences among multiple groups are analyzed by one-way ANOVA with an appropriate post hoc test. A two-way ANOVA or mixed-effects model with an appropriate post hoc test is applied to analyze the effects of multiple factors. Differences between two groups are analyzed using the Student's t-test or Mann-Whitney U-test. In addition, the results brain delivery and M/MΦ targeting are correlated with therapeutic outcomes, which serves as the basis for using imaging as guidance for therapeutic treatment. All analysis are carried out with GraphPad prism 10 software. Differences with P<0.05 are considered to be significant.

An eEV-IL10-based therapy with a higher treatment efficacy than naked IL-10, PSL-IL10, and EVs alone (justified by P<0.05) is developed. The therapy has a strong correlation (R2>0.7) between the quantity of eEV-IL10 delivered to the affected brain region and improvement in therapeutic outcome.

Improved outcomes are observed when treatment starts at 30 min and 2 h post-ICH. For treatment starting 1 day or 3 days post-ICH, if no significant improvements are seen, the duration of treatment is extended (i.e., 14 days) until significant outcomes (assessed by MRI) are achieved.

### Rigorous Experimental Design for Robust and Unbiased Results:

2-month-old mice of both genders are used to evaluate treatment outcomes. All in vivo experiments are performed in accordance with the STAIR and RIGOR guidelines. Animals that die during surgery or before treatment are excluded from further studies. All measurements are conducted by an investigator blinded to group identity. All results (negative and positive), a priori exclusion/inclusion criteria, and power/sample size calculations are reported.

Using a 80% power for detecting differences in the neurologic deficits and lesion volume, in one aspect, power analysis determines the appropriate cohort size to be 8 mice per group, or 10 mice considering ˜85% survival in 2-month-old mice. The statistical comparisons among multiple groups (treatment vs. vehicle; behavior tests at different time points) are made by ANOVA (one-way, two-way, or repeated measures depending on the number of variables) followed by Bonferroni/Dunn post hoc correction. Differences between two groups are determined by a two-tailed Student's t-test. Covariance is analyzed by multiple regression models. For data not normally distributed, nonparametric tests are used. Statistical significance is set at p<0.05.

### LIST OF REFERENCES

Achmus, L. et al. Stroke-Induced Modulation of Myeloid-Derived Suppressor Cells (MDSCs) and IL-10-Producing Regulatory Monocytes. Front Neurol 11, 577971, doi:10.3389/fneur.2020.577971 (2020).
Alvarez-Erviti, L. et al. Delivery of siRNA to the mouse brain by systemic injection of targeted exosomes. Nat Biotechnol 29, 341-345, doi:10.1038/nbt.1807 (2011).
Arabpour, M., Saghazadeh, A. & Rezaei, N. Anti-inflammatory and M2 macrophage polarization-promoting effect of mesenchymal stem cell-derived exosomes. Int Immunopharmacol. 2021; 97:107823.
Arjmand, B. et al. (Springer US, 2023).
Bang, O. Y. & Kim, E. H. Mesenchymal Stem Cell-Derived Extracellular Vesicle Therapy for Stroke: Challenges and Progress. Front Neurol 10, 211, doi:10.3389/fneur.2019.00211 (2019).
Barrett, J. P. et al. NOX2 deficiency alters macrophage phenotype through an IL-10/STAT3 dependent mechanism: implications for traumatic brain injury. J Neuroinflammation 14, 65, doi:10.1186/s12974-017-0843-4 (2017).
Bi, Y. et al. Systemic proteomics and miRNA profile analysis of exosomes derived from human pluripotent stem cells. Stem Cell Res Ther 13, 449, doi:10.1186/s13287-022-03142-1(2022).
Borger, V. et al. Mesenchymal Stem/Stromal Cell-Derived Extracellular Vesicles and Their Potential as Novel Immunomodulatory Therapeutic Agents. Int J Mol Sci 18, doi:10.3390/ijms18071450 (2017).
Chen, Z. et al. CT and CEST MRI bimodal imaging of the intratumoral distribution of iodinated liposomes. Quant Imaging Med Surg 9, 1579-1591, doi:10.21037/qims.2019.06.10 (2019).
Chou, B. K., Mali, P., Huang, X., Ye, Z., Dowey, S. N., Resar, L. M., Zou, C., Zhang, Y. A., Tong, J. & Cheng, L. Efficient human iPS cell derivation by a non-integrating plasmid from blood cells with unique epigenetic and gene expression signatures. Cell Res. 2011; 21(3):518-529.
de Oliveira Manoel, A. L. Surgery for spontaneous intracerebral hemorrhage. Crit Care 24, 45, doi:10.1186/s13054-020-2749-2 (2020).
Driedonks, T. et al. Pharmacokinetics and biodistribution of extracellular vesicles administered intravenously and intranasally to Macaca nemestrina. J Extracell Biol 1, doi:10.1002/jex2.59 (2022).
Erdo, F., Bors, L. A., Farkas, D., Bajza, A. & Gizurarson, S. Evaluation of intranasal delivery route of drug administration for brain targeting. Brain Res Bull. 2018; 143:155-170.
Fedorak, R. N., Gangl, A., Elson, C. O., Rutgeerts, P., Schreiber, S., Wild, G., Hanauer, S. B., Kilian, A., Cohard, M., LeBeaut, A. & Feagan, B. Recombinant human interleukin 10 in the treatment of patients with mild to moderately active Crohn's disease. The Interleukin 10 Inflammatory Bowel Disease Cooperative Study Group. Gastroenterology. 2000; 119(6):1473-1482.
Feigin, V. L. et al. Global, regional, and national burden of stroke and its risk factors, 1990-2019: a systematic analysis for the Global Burden of Disease Study 2019. The Lancet Neurology 20, 795-820, doi:10.1016/s1474-4422(21)00252-0 (2021).
Fisher, M. et al. Update of the stroke therapy academic industry roundtable preclinical recommendations. Stroke 40, 2244-2250, doi:10.1161/STROKEAHA.108.541128 (2009).
Hagimori, M. et al. Synthesis of high functionality and quality mannose-grafted lipids to produce macrophage-targeted liposomes. Eur J Pharm Sci 123, 153-161, doi:10.1016/j.ejps.2018.07.036 (2018).
Han, X., Lan, X., Li, Q., Gao, Y., Zhu, W., Cheng, T., Maruyama, T. & Wang, J. Inhibition of prostaglandin E2 receptor EP3 mitigates thrombin-induced brain injury. J Cereb Blood Flow Metab. 2016; 36(6):1059-1074.
Han, Y. et al. Multipotent mesenchymal stromal cell-derived exosomes improve functional recovery after experimental intracerebral hemorrhage in the rat. J Neurosurg 131, 290-300, doi:10.3171/2018.2. JNS171475 (2018).
Han, Z. et al. Highly efficient magnetic labelling allows MRI tracking of the homing of stem cell-derived extracellular vesicles following systemic delivery. J Extracell Vesicles 10, e12054, doi:10.1002/jev2.12054 (2021).
Hanley, D. F. et al. Efficacy and safety of minimally invasive surgery with thrombolysis in intracerebral haemorrhage evacuation (MISTIE III): a randomised, controlled, open-label, blinded endpoint phase 3 trial. Lancet 393, 1021-1032, doi:10.1016/50140-6736(19)30195-3 (2019).
Herrmann, I. K., Wood, M. J. A. & Fuhrmann, G. Extracellular vesicles as a next-generation drug delivery platform. Nature nanotechnology. 2021; 16(7):748-759.
Hinojosa, A. E., Garcia-Bueno, B., Leza, J. C. & Madrigal, J. L. CCL2/MCP-1 modulation of microglial activation and proliferation. J Neuroinflammation. 2011; 8:77.
Hu, L., Wickline, S. A. & Hood, J. L. Magnetic resonance imaging of melanoma exosomes in lymph nodes. Magnetic resonance in medicine 74, 266-271, doi:10.1002/mrm.25376 (2015).
Jiang, C. et al. Immune changes in peripheral blood and hematoma of patients with intracerebral hemorrhage. FASEB J 34, 2774-2791, doi:10.1096/fj.201902478R (2020).
Jiang, L. et al. A post-insertion strategy for surface functionalization of bacterial and mammalian cell-derived extracellular vesicles. Biochim Biophys Acta Gen Subj 1865, 129763, doi:10.1016/j.bbagen.2020.129763 (2021).
Karpman, D., Stahl, A. L. & Arvidsson, I. Extracellular vesicles in renal disease. Nature reviews. Nephrology 13, 545-562, doi:10.1038/nrneph.2017.98 (2017).
Lan, X., Han, X., Li, O., Yang, O. W. & Wang, J. Modulators of microglial activation and polarization after intracerebral haemorrhage. Nat Rev Neurol 13, 420-433, doi:10.1038/nrneurol.2017.69 (2017).
Lan, X., Liu, R., Sun, L., Zhang, T. & Du, G. Methyl salicylate 2-O-beta-D-lactoside, a novel salicylic acid analogue, acts as an anti-inflammatory agent on microglia and astrocytes. J Neuroinflammation 8, 98, doi:10.1186/1742-2094-8-98 (2011).
Lapchak, P. A., Zhang, J. H. & Noble-Haeusslein, L. J. RIGOR guidelines: escalating STAIR and STEPS for effective translational research. Transl Stroke Res 4, 279-285, doi:10.1007/s12975-012-0209-2 (2013).
Laso-Garcia, F. et al. Circulating extracellular vesicles promote recovery in a preclinical model of intracerebral hemorrhage. Molecular Therapy—Nucleic Acids 32, 247-262, doi:10.1016/j.omtn.2023.03.006 (2023).
Li, Q. et al. Inhibition of neuronal ferroptosis protects hemorrhagic brain. JCI Insight 2, e90777, doi:10.1172/jci.insight.90777 (2017).
Li, Q. et al. Inhibition of tPA-induced hemorrhagic transformation involves adenosine A2b receptor activation after cerebral ischemia. Neurobiol Dis 108, 173-182, doi:10.1016/j.nbd.2017.08.011 (2017).
Li, Q. et al. Microglia-derived interleukin-10 accelerates post-intracerebral hemorrhage hematoma clearance by regulating CD36. Brain Behav Immun 94, 437-457, doi:10.1016/j.bbi.2021.02.001 (2021).
Li, Q. et al. Neuroprotection of brain-permeable iron chelator VK-28 against intracerebral hemorrhage in mice. J Cereb Blood Flow Metab 37, 3110-3123, doi:10.1177/0271678X17709186 (2017).
Li, Q. et al. Expression of Tmeml19/Sall1 and Ccr2/CD69 in FACS-Sorted Microglia- and Monocyte/Macrophage-Enriched Cell Populations After Intracerebral Hemorrhage. Front Cell Neurosci 12, 520, doi:10.3389/fncel.2018.00520 (2018).
Li, Q. et al. Organotypic Hippocampal Slices as Models for Stroke and Traumatic Brain Injury. Mol Neurobiol. 2016; 53(6):4226-4237.
Li, Q., Han, X., Lan, X., Gao, Y., Wan, J., Durham, F., Cheng, T., Yang, J., Wang, Z., Jiang, C., Ying, M., Koehler, R. C., et al. Inhibition of neuronal ferroptosis protects hemorrhagic brain. JCI Insight. 2017; 2(7):e90777.
Li, Q., Han, X., Lan, X., Hong, X., Li, Q., Gao, Y., Luo, T., Yang, Q., Koehler, R. C., Zhai, Y., Zhou, J. & Wang, J. Inhibition of tPA-induced hemorrhagic transformation involves adenosine A2b receptor activation after cerebral ischemia. Neurobiol Dis. 2017; 108:173-182.
Li, Q., Lan, X., Han, X. & Wang, J. Expression of Tmeml19/Sall1 and Ccr2/CD69 in FACS-Sorted Microglia- and Monocyte/Macrophage-Enriched Cell Populations After Intracerebral Hemorrhage. Front Cell Neurosci. 2018; 12:520.
Li, Q., Lan, X., Han, X., Durham, F., Wan, J., Weiland, A., Koehler, R. C. & Wang, J. Microglia-derived interleukin-10 accelerates post-intracerebral hemorrhage hematoma clearance by regulating CD36. Brain Behav Immun. 2021. DOI:10.1016/j.bbi.2021.02.001.
Li, Q., Wan, J., Lan, X., Han, X., Wang, Z. & Wang, J. Neuroprotection of brain-permeable iron chelator VK-28 against intracerebral hemorrhage in mice. J Cereb Blood Flow Metab. 2017; 37(9):3110-3123.
Li, Y. et al. CEST theranostics: label-free MR imaging of anticancer drugs. Oncotarget 7, 6369-6378, doi:10.18632/oncotarget.7141 (2016).
Li, Y., Qiao, Y., Chen, H., Bai, R., Staedtke, V., Han, Z., Xu, J., Chan, K. W. Y., Yadav, N., Bulte, J. W. M., Zhou, S., van Zijl, P. C. M., et al. Characterization of tumor vascular permeability using natural dextrans and CEST MRI. Magn Reson Med. 2018; 79(2):1001-1009.
Liesz, A., Bauer, A., Hoheisel, J. D. & Veltkamp, R. Intracerebral interleukin-10 injection modulates post-ischemic neuroinflammation: an experimental microarray study. Neurosci Lett 579, 18-23, doi:10.1016/j.neulet.2014.07.003 (2014).
Liu, G. et al. In vivo multicolor molecular MR imaging using diamagnetic chemical exchange saturation transfer liposomes. Magn Reson Med 67, 1106-1113, doi:10.1002/mrm.23100 (2012).
Liu, H. et al. Label-free CEST MRI Detection of Citicoline-Liposome Drug Delivery in Ischemic Stroke. Theranostics 6, 1588-1600, doi:10.7150/thno.15492 (2016).
Liu, S., Mahairaki, V., Bai, H., Ding, Z., Li, J., Witwer, K. W. & Cheng, L. Highly Purified Human Extracellular Vesicles Produced by Stem Cells Alleviate Aging Cellular Phenotypes of Senescent Human Cells. Stem Cells. 2019; 37(6):779-790.
Liu, X.-S., Chopp, M., Li, C., Pan, W.-L., Fan, B., Levin, A. M., Zhang, R. & Zhang, Z. Comprehensive proteomics and microRNA analyses of adult neural stem cell derived exosomes after stroke. 2019.
Maiti, P. et al. Transplantation of mesenchymal stem cells overexpressing interleukin-10 induces autophagy response and promotes neuroprotection in a rat model of TBI. J Cell Mol Med 23, 5211-5224, doi:10.1111/jcmm.14396 (2019).
Matsumoto, J., Stewart, T., Banks, W. A. & Zhang, J. The transport mechanism of extracellular vesicles at the blood-brain barrier. Current pharmaceutical design. 2017; 23(40):6206-6214.
Mendelow, A. D. et al. Early surgery versus initial conservative treatment in patients with spontaneous supratentorial intracerebral haematomas in the International Surgical Trial in Intracerebral Haemorrhage.
Mendelow, A. D., Gregson, B. A., Rowan, E. N., Murray, G. D., Gholkar, A., Mitchell, P. M. & Investigators, S. I. Early surgery versus initial conservative treatment in patients with spontaneous supratentorial lobar intracerebral haematomas (STICH II): a randomised trial. Lancet. 2013; 382(9890):397-408.
Murray, P. J. The primary mechanism of the IL-10-regulated antiinflammatory response is to selectively inhibit transcription. Proc Natl Acad Sci USA. 2005; 102(24):8686-8691.
Ng, C. Y. et al. Scalable Production of Extracellular Vesicles and Its Therapeutic Values: A Review. International Journal of Molecular Sciences 23, 7986, doi:10.3390/ijms23147986 (2022).
Ni, W., Ramalingam, M., Li, Y., Park, J.-H., Dashnyam, K., Lee, J.-H., Bloise, N., Fassina, L., Visai, L. & De Angelis, M. G. C. Immunomodulatory and Anti-inflammatory effect of Neural Stem/Progenitor Cells in the Central Nervous System. Stem Cell Reviews and Reports. 2023:1-20.
Patel, S. et al. In vivo tracking of [89Zr]Zr-labeled engineered extracellular vesicles by PET reveals organ-specific biodistribution based upon the route of administration. Nuclear Medicine and Biology 112-113, 20-30, doi:10.1016/j.nucmedbio.2022.06.004 (2022).
Phan, J. et al. Engineering mesenchymal stem cells to improve their exosome efficacy and yield for cell-free therapy. Journal of extracellular vesicles 7, 1522236 (2018).
Piffoux, M., Silva, A. K. A., Wilhelm, C., Gazeau, F. & Tareste, D. Modification of Extracellular Vesicles by Fusion with Liposomes for the Design of Personalized Biogenic Drug Delivery Systems. ACS Nano 12, 6830-6842, doi:10.1021/acsnano.8b02053 (2018).
Radwanski, E., Chakraborty, A., Van Wart, S., Huhn, R. D., Cutler, D. L., Affrime, M. B. & Jusko, W. J. Pharmacokinetics and leukocyte responses of recombinant human interleukin-10. Pharm Res. 1998; 15(12):1895-1901.
Rohde, E., Pachler, K. & Gimona, M. Manufacturing and characterization of extracellular vesicles from umbilical cord-derived mesenchymal stromal cells for clinical testing. Cytotherapy 21, 581-592, doi:10.1016/j.jcyt.2018.12.006 (2019).
Saint-Pol, J., Gosselet, F., Duban-Deweer, S., Pottiez, G. & Karamanos, Y. Targeting and Crossing the Blood-Brain Barrier with Extracellular Vesicles. Cells 9, 851, doi:10.3390/cells9040851 (2020).
Santoso, M. R. & Yang, P. C. Molecular Imaging of Stem Cells and Exosomes for Myocardial Regeneration. Curr Cardiovasc Imag 10, doi:10.1007/s12410-017-9433-1 (2017).
Saxena, A., Khosraviani, S., Noel, S., Mohan, D., Donner, T. & Hamad, A. R. Interleukin-10 paradox: A potent immunoregulatory cytokine that has been difficult to harness for immunotherapy. Cytokine. 2015; 74(1):27-34.
Schrag, M. & Kirshner, H. Management of Intracerebral Hemorrhage: JACC Focus Seminar. J Am Coll Cardiol 75, 1819-1831, doi:10.1016/j.jacc.2019.10.066 (2020).
Sordi, R., Bet, A. C., Della Justina, A. M., Ramos, G. C. & Assreuy, J. The apoptosis clearance signal phosphatidylserine inhibits leukocyte migration and promotes inflammation resolution in vivo. Eur J Pharmacol 877, 173095, doi:10.1016/j.ejphar.2020.173095 (2020).
Struck, D. K., Hoekstra, D. & Pagano, R. E. Use of resonance energy transfer to monitor membrane fusion. Biochemistry 20, 4093-4099, doi:10.1021/bi00517a023 (1981).
Tamo, L., Gazdhar, A., Feki, A. & Geiser, T. The secretome of induced pluripotent stem cells (iPSC) modulates macrophage phenotype in the fibrotic lung. (Eur Respiratory Soc, 2015).
Taylor, R. A. et al. TGF-beta1 modulates microglial phenotype and promotes recovery after intracerebral hemorrhage. J Clin Invest 127, 280-292, doi:10.1172/JCI88647 (2017).
Thery, C., Witwer, K. W., Aikawa, E., Alcaraz, M. J., Anderson, J. D., Andriantsitohaina, R., Antoniou, A., Arab, T., Archer, F., Atkin-Smith, G. K., Ayre, D. C., Bach, J. M., et al. Minimal information for studies of extracellular vesicles 2018 (MISEV2018): a position statement of the International Society for Extracellular Vesicles and update of the MISEV2014 guidelines. J Extracell Vesicles. 2018; 7(1):1535750.
Toita, R., Kawano, T., Murata, M. & Kang, J. H. Anti-obesity and anti-inflammatory effects of macrophage-targeted interleukin-10-conjugated liposomes in obese mice. Biomaterials 110, 81-88, doi:10.1016/j.biomaterials.2016.09.018 (2016).
Upadhya, R., Madhu, L. N., Attaluri, S., Gitai, D. L. G., Pinson, M. R., Kodali, M., Shetty, G., Zanirati, G., Kumar, S. & Shuai, B. Extracellular vesicles from human iPSC-derived neural stem cells: miRNA and protein signatures, and anti-inflammatory and neurogenic properties. Journal of extracellular vesicles. 2020; 9(1):1809064.
Wang, X., Wong, K., Ouyang, W. & Rutz, S. Targeting IL-10 Family Cytokines for the Treatment of Human Diseases. Cold Spring Harb Perspect Biol. 2019; 11(2).
Wolfram, J. & Ferrari, M. Clinical Cancer Nanomedicine. Nano Today. 2019; 25:85-98.
Xu, X. et al. Anti-inflammatory and immunomodulatory mechanisms of atorvastatin in a murine model of traumatic brain injury. J Neuroinflammation 14, 167, doi:10.1186/s12974-017-0934-2 (2017).
Xu, Z., Tian, N., Li, S., Li, K., Guo, H., Zhang, H., Jin, H., An, M. & Yu, X. Extracellular vesicles secreted from mesenchymal stem cells exert anti-apoptotic and anti-inflammatory effects via transmitting microRNA-18b in rats with diabetic retinopathy. International immunopharmacology. 2021; 101:108234.
Xue, M. & Yong, V. W. Neuroinflammation in intracerebral haemorrhage: immunotherapies with potential for translation. Lancet Neurol 19, 1023-1032, doi:10.1016/51474-4422(20)30364-1(2020).
Yang, G. et al. Mesenchymal Stem Cell Application and Its Therapeutic Mechanisms in Intracerebral Hemorrhage. Front Cell Neurosci 16, 898497, doi:10.3389/fncel.2022.898497 (2022).
Yang, J. et al. Multimodality MRI assessment of grey and white matter injury and blood-brain barrier disruption after intracerebral haemorrhage in mice. Sci Rep 7, 40358, doi:10.1038/srep40358 (2017).
Yin, L. et al. Therapeutic Advances of Stem Cell-Derived Extracellular Vesicles in Regenerative Medicine. Cells 9, doi:10.3390/cells9030707 (2020).
Yu, Y. et al. Genetically engineered exosomes display RVG peptide and selectively enrich a neprilysin variant: a potential formulation for the treatment of Alzheimer's disease. J Drug Target 29, 1128-1138, doi:10.1080/1061186X.2021.1929257 (2021).
Zhang, Z. G., Buller, B. & Chopp, M. Exosomes—beyond stem cells for restorative therapy in stroke and neurological injury. Nat Rev Neurol. 2019; 15(4):193-203.
Zhou, J.-f. et al. Application of stem cells and exosomes in the treatment of intracerebral hemorrhage: an update. Stem Cell Research & Therapy 13, 281, doi:10.1186/s13287-022-02965-2 (2022).
Zhu, W. et al. Changes in motor function, cognition, and emotion-related behavior after right hemispheric intracerebral hemorrhage in various brain regions of mouse. Brain Behav Immun 69, 568-581, doi:10.1016/j.bbi.2018.02.004 (2018).
Zou, Y. et al. Mesenchymal stem cell-derived extracellular vesicles/exosome: A promising therapeutic strategy for intracerebral hemorrhage. Regenerative Therapy 22, 181-190, doi:10.1016/j.reth.2023.01.006 (2023).

Although the invention has been described with reference to the above examples, it will be understood that modifications and variations are encompassed within the spirit and scope of the invention. Accordingly, the invention is limited only by the following claims.

What is claimed is:

1. A composition comprising:
a stem cell derived extracellular vesicle (SC-EV); and
a liposome comprising a therapeutic agent.

2. The composition of claim 1, wherein the liposome comprises at least one of phosphatidylcholine or phosphatidylserine.

3. The composition of claim 1, wherein the liposome is a phosphatidylserine (PS) liposome.

4. The composition of claim 1, wherein the stem cell is a mesenchymal stem cell (MSC), a hematopoietic stem cell (HSC), an induced pluripotent stem cell (iPSC), an adipose tissue derived stem cell, or a neural stem cell (NSC).

5. The composition of claim 1, wherein the therapeutic agent is a cytokine, an immunomodulatory agent, an antioxidant, an miRNA, or an antibody.

6. The composition of claim 5, wherein the cytokine is interleukin-1 (IL-1), interleukin-2 (IL-2), interleukin-6 (IL-6), interleukin-10 (IL-10), interleukin-12 (IL-12), interleukin-15 (IL-15), interleukin-18 (IL-18), tumor necrosis factors (TNF), interferons (IFN), colony-stimulating factors (GM-CSF) or transforming growth factor-β (TGF-β).

7. The composition of claim 5, wherein the immunomodulatory agent is a checkpoint inhibitor, a small molecule, or a chimeric antigen receptor.

8. The composition of claim 5, wherein the antibody is adalimumab, infliximab, certolizumab, golimumab, or tocilizumab.

9. The composition of claim 1, wherein the liposome further comprises an imaging agent.

10. The composition of claim 9, wherein the imaging agent is selected from superparamagnetic iron oxide (SPIO) nanoparticles, a lipid conjugated metal chelator, a radionucleotide, a fluorescent protein or a lipophilic fluorescence dye.

11. The composition of claim 1, further comprising a pharmaceutically acceptable carrier.

12. A method of generating a chimeric extracellular vesicle (cEV) comprising fusing a stem cell derived extracellular vesicle (SC-EV) and a liposome comprising a therapeutic agent, thereby generating a cEV.

13. The method of claim 12, wherein the liposome comprises at least one of phosphatidylcholine or phosphatidylserine.

14. The method of claim 12, wherein the liposome is a phosphatidylserine (PS) liposome.

15. The method of claim 12, wherein the stem cell is a mesenchymal stem cell (MSC), a hematopoietic stem cell (HSC), an induced pluripotent stem cell (iPSC), an adipose tissue derived stem cell, or a neural stem cell (NSC).

16. The method of claim 12, wherein the therapeutic agent is a cytokine, an immunomodulatory agent, an antioxidant, an miRNA, or an antibody.

17. The method of claim 16, wherein the cytokine is interleukin-1 (IL-1), interleukin-2 (IL-2), interleukin-6 (IL-6), interleukin-10 (IL-10), interleukin-12 (IL-12), interleukin-15 (IL-15), interleukin-18 (IL-18), tumor necrosis factors (TNF), interferons (IFN), colony-stimulating factors (GM-CSF) or transforming growth factor-β (TGF-β).

18. The method of claim 16, wherein the immunomodulatory agent is a checkpoint inhibitor, a small molecule, or a chimeric antigen receptor.

19. The method of claim 16, wherein the antibody is adalimumab, infliximab, certolizumab, golimumab, or tocilizumab.

20. The method of claim 12, wherein the liposome further comprises an imaging agent.

21. The method of claim 20, wherein the imaging agent is selected from superparamagnetic iron oxide (SPIO) nanoparticles, a lipid conjugated metal chelator, a radionucleotide, a fluorescent protein or a lipophilic fluorescence dye.

22. A method of treating inflammation comprising administering the composition of claim 1 to a subject in need thereof.

23. The method of claim 22, wherein the inflammation is neuroinflammation.

24. The method of claim 23, wherein the subject has an intracerebral hemorrhage (ICH).

25. The method of claim 22, wherein the administering is systemic, intranasal, intrathecal or intracerebral.

26. A method of detecting or guiding cEV-based therapy comprising: administering a composition of claim 1 to a subject and detecting the composition after administration.

27. The method of claim 26, wherein the detecting is by magnetic particle imaging (MPI), positron emission tomography (PET), fluorescence imaging (FI) or bioluminescence imaging (BLI).

28. The method of claim 26, wherein the detecting provides information for optimization of the cEV-based therapy.
